(19) United States
(12) Patent Application Publication
(10) Pub. No.: US 2026/0183549 A1
(43) Pub. Date: Jul. 2, 2026

(54) TREATING OR PREVENTING ANOREXIA NERVOSA VIA PRECISION TARGETING OF NEURONAL CIRCUIT

(71) Applicant: ARIZONA BOARD OF REGENTS ON BEHALF OF THE UNIVERSITY OF ARIZONA, Tucson, AZ (US)

(72) Inventors: Haijiang CAI, Tucson, AZ (US); Wesley SCHNAPP, Tucson, AZ (US)

(21) Appl. No.: 19/124,301

(22) PCT Filed: Nov. 2, 2023
(86) PCT No.: PCT/US2023/078488

§ 371 Date: Apr. 25, 2025

Related U.S. Application Data
(60) Provisional application No. 63/382,049, filed on Nov. 2, 2022.

(51) Int. Cl. A61N 1/36; A61K 45/06; A61P 25/00

(52) U.S. Cl. CPC A61N 1/36085; A61K 45/06; A61P 25/00

(57) ABSTRACT

Methods of treating one or more eating disorders including anorexia nervosa, bulimia, and related clinical syndromes in a subject in need thereof are described. In some cases, electric stimulation and/or one or more active agents are administered to a subject in need thereof to reduce or inhibit the activity of CeA PKC-δ neurons and ovBNST PKC-δ neurons in the brain of the subject. Preferably, the disclosed methods are effective to increase or excite the activity of the brain regions downstream of CeA PKC-δ neurons and ovBNST PKC-δ neurons including medial part of CeA and ventral lateral part of BNST.

### CROSS-REFERENCE TO RELATED APPLICATION

This application claims the benefit of and priority to U.S. Ser. No. 63/382,049 filed Nov. 2, 2022 and which is incorporated by referenced herein in its entirety.

### STATEMENT REGARDING FEDERALLY SPONSORED RESEARCH

This invention was made with government support under Grant No. R01 DK124501 awarded by the National Institutes of Health. The government has certain rights in the invention.

### FIELD OF THE INVENTION

The invention is generally in the field of treatment for eating disorders, such as anorexia nervosa and bulimia nervosa, and related clinical syndromes.

### BACKGROUND OF THE INVENTION

Anorexia nervosa (AN) is a prevalent eating disorder seen primarily in females that significantly disrupts life and health, to the point of reaching fatality in extreme cases, with the highest mortality rate of any psychiatric disorder (Arcelus, J., et al., Arch Gen Psychiatry, 2011. 68(7): p. 724-31; Jagielska, G. and I. Kacperska, Psychiatr Pol, 2017. 51(2): p. 205-218.). AN is characterized by self-starvation, fear of gaining weight, and excessive exercise, but also is often co-diagnosed with other psychiatric and emotional disorders, such as depression, anxiety, and obsessive compulsive disorder (American Psychiatric Association, Desk reference to the diagnostic criteria from DSM-5. 2013, Washington, DC: American Psychiatric Publishing. xlviii, 395 p; Guarda, A. S., et al., Physiol Behav, 2015. 152(Pt B): p. 466-72; Hebebrand, J., et al., Physiol Behav, 2003. 79(1): p. 25-37; Kron, L., et al., Compr Psychiatry, 1978. 19(5): p. 433-40; Mattar, L., et al., Psychiatry Res, 2012. 200(2-3): p. 513-7; and Zipfel, S., et al., Lancet Psychiatry, 2015. 2(12): p. 1099-111.). These characteristics of AN suggest the neural circuits regulating eating behavior and the neural circuits regulating emotion interact extensively and significantly to control AN development.

It has been proposed that the primary neural dysfunction responsible for activity-based anorexia (ABA) development is centered on reward systems (Beeler, J. A. and N. S. Burghardt, J Exp Neurol, 2021. 2(1): p. 21-28; O'Hara, C. B., I. C. Campbell, and U. Schmidt, Neurosci Biobehav Rev, 2015. 52: p. 131-52). This theory has been emphasized and supported by several studies. For example, subcutaneous infusion of a low dose of non-selective dopaminergic antagonist attenuates ABA while a high dose exacerbates it (Verhagen, L. A., et al., Eur Neuropsychopharmacol, 2009. 19(3): p. 153-60). Similarly, administration of D2/3 antagonists ameliorates the ABA phenotypes in mice (Klenotich, S. J., et al., Transl Psychiatry, 2015. 5: p. e613). These results are consistent with a recent study demonstrating that a D2 to D1 shift in dopaminergic pathway regulates ABA, and that interruption or inhibition of the dopamine signaling pathways attenuates ABA (Cai, X., et al., Nat Neurosci, 2022. 25(5): p. 646-658). However, these manipulations of the dopamine pathways tend to have only a mild attenuation effect on ABA development, with majority of the animals still developing ABA but in a delayed time. Surprisingly, a small—although not significant—increase in FAA is observed after infusion of dopaminergic antagonist (Verhagen, L. A., et al., Eur Neuropsychopharmacol, 2009. 19(3): p. 153-60), which is, in fact, contradictory to being less susceptible to developing ABA. Involvement of the dopamine pathway in ABA development is also supported by an experiment showing that overexpression of dopamine receptor in nucleus accumbens (NAc) induces more weight loss, reduced food intake, and increased wheel activity during ABA (Welch, A. C., et al., Mol Psychiatry, 2021. 26(8): p. 3765-3777). Chemogenetic excitation of the rewarding pathway from ventral tegmental area (VTA) to NAc pathway in female rats attenuates weight loss, increases food intake, yet has no effect on overall running activity, but rather, a surprising increase in FAA (Foldi, C. J., L. K. Milton, and B. J. Oldfield, Neuropsychopharmacology, 2017. 42(12): p. 2292-2300). Furthermore, using a mild ABA development rat model, in which ˜40% of the animals develop ABA, a study showed chemogenetic silencing of the pathway from medial prefrontal cortex to NAc prevents ABA development (Milton, L. K., et al., Biol Psychiatry, 2021. 90(12): p. 819-828). However, this manipulation has little to no effect on food intake, does not impact overall running wheel activity and, again, causes a surprising increase in FAA. These studies suggest that the reward pathway may play a role in regulating ABA, but does not directly contribute to ABA development as the effect on preventing ABA is mild with some characteristics in ABA not being affected, or even being affected in the opposite direction.

Another promising neural circuit that might contribute to ABA development is that in the hypothalamus region, which has been well-studied and established as regulating feeding behavior, metabolism, and energy balance (Sternson, S. M. and A. K. Eiselt, Annu Rev Physiol, 2017. 79: p. 401-423; Andermann, M. L. and B. B. Lowell, Neuron, 2017. 95(4): p. 757-778; Watts, A. G., et al., Physiol Rev, 2022. 102(2): p. 689-813). For example, activation of neurons expressing agouti-related protein (AgRP) in the arcuate nucleus promotes eating behaviors. However, a recent study found that activation of the AgRP neurons makes no difference in rescuing the decreased body weight or caloric intake, but allowed mice to sustain the increased wheel activity before physical exhaustion in the regular ABA paradigm (Miletta, M. C., et al., Nat Metab, 2020. 2(11): p. 1204-1211), suggesting alteration of the AgRP neuron activity does not contribute to ABA development. It remains to be determined if other neural pathways in this canonical eating center regulate ABA development.

Thus, the underlying neural mechanisms involved in AN, particularly how neurons in the amygdala—the most well-established region for emotion control—regulate the development of AN, remain to be determined. There remains an urgent need for an effective intervention strategy in treating eating disorders.

It is an object of the invention to provide compositions and methods for treating one or more eating disorders including anorexia nervosa, bulimia, and related clinical syndromes in a subject.

It is also another object of the invention to provide safe and efficacious compositions and methods for treating one or more eating disorders with minimal toxicity.

### SUMMARY OF THE INVENTION

Methods of treating or preventing one or more eating disorders including anorexia nervosa and bulimia, and related clinical syndromes in a subject in need thereof are described.

It has been established that simultaneous ablation of a specific subpopulation of neurons, marked by the expression of protein kinase C-delta (PKC-δ), in two nuclei of the central extended amygdala (EAc) rescues all three key phenotypes of the activity-based anorexia (ABA) animal model: extreme increases in wheel activity, decreases in food intake, and, consequently, life-threatening loss in body weight.

Thus, methods include administering to a subject in need thereof an effective amount of electric stimulation to reduce or inhibit the activity of CeA PKC-δ neurons and ovBNST PKC-δ neurons in the brain of the subject, preferably, in an amount effective to increase or excite the activity of the brain regions downstream of CeA PKC-δ neurons and ovBNST PKC-δ neurons such as medial part of CeA and ventral lateral part of BNST.

Methods also include administering to a subject in need thereof an effective amount of a first pharmacological drug to reduce or inhibit the activity of CeA PKC-δ neurons and ovBNST PKC-δ neurons in the brain of the subject. In some embodiments, the first pharmacological drug can inhibit the activity of CeA PKC-δ neurons or ovBNST PKC-δ neurons, or both. In other embodiments, the first pharmacological drug inhibits the activity of CeA PKC-δ neurons, and wherein the methods further include administering a second pharmacological drug that inhibits the activity of ovBNST PKC-δ neurons. The first pharmacological drug and the second pharmacological drug are administered simultaneously or sequentially. Typically, the first pharmacological drug is administered locally to the brain, optionally, the pharmacological drug is administered locally to the CeA PKC-δ neurons and/or ovBNST PKC-δ neurons in the brain. Preferably, the first pharmacological drug and/or the second pharmacological drug are administered in an amount effective to increase or excite the activity of the brain regions downstream of CeA PKC-δ neurons and ovBNST PKC-δ neurons including medial part of CeA and/or ventral lateral part of BNST.

### BRIEF DESCRIPTION OF THE DRAWINGS

FIGS. 1A-1B are bar graphs showing the number of average density of CeA^{PKC-δ+} neurons (number of neurons per micron×10⁴) in mouse brains after bilateral injections of a Cre-dependent virus expressing caspase into the CeA regions of mouse brains of WT (PKC-δ⁺ neurons intact) and PKC-δ-Cre (PKC-δ⁺ neurons ablated) (FIG. 1A); and the number of average density of ovBNST^{PKC-δ+} neurons (number of neurons per micron×10⁴) in mouse brains after bilateral injections of a Cre-dependent virus expressing caspase into the ovBNST regions of mouse brains of WT (PKC-δ⁺ neurons intact) and PKC-δ-Cre (PKC-δ+ neurons ablated) (FIG. 1B). Unpaired t-tests: CeA, p=2.2×10⁻¹³, WT n=8, PKC-δ-Cre n=20; ovBNST, p=8.9×10⁻¹⁶, WT n=12, PKC-δ-Cre n=19.

FIGS. 2A-2B are a schematic diagram showing timeline of ABA protocol (created with BioRender.com) (FIG. 2A); and a survival analysis of different experimental mice (FIG. 2B). With respect to FR (n=18): FRW WT/no ablation (n=10, p=6×10⁻⁷), FRW-Cre CeA^{PKC-δ} ablation (n=5, p=0.05), FRW-Cre ovBNST^{PKC-δ} ablation (n=5, p=0.008), and FRW-Cre OVBNST^{PKC-δ}+CeA^{PKC-δ} ablation (n=10, p=0.5). The dashed lines specify the median survival for respective groups. BLA: basolateral amygdala, AC: anterior commissure, FR: food restricted (includes mice from each ablation group), FRW: food restricted with wheel. All samples are females. *p<0.05, **p<0.01, ***p<0.001.

FIGS. 3A-3H are graphs showing percentage of mean body weight loss across 10 days of food restriction (FIG. 3A) in FR (▴) and FRW (∘) no ablation (WT) groups, and percentage of body weight loss on the day of removal from ABA experiment, either when the ABA criteria is reached (20% loss from baseline weight two days in a row) or after day 10 (FIG. 3B) in FR and FRW no ablation (WT) groups; graphs showing percentage of body weight loss across 10 days of food restriction (FIG. 3C) in FR (▴) and FRW (∘) PKC-δ-Cre mice (dual ablation), and percentage of body weight loss on the day of removal from ABA experiment, either when the ABA criteria is reached (20% loss from baseline weight two days in a row) or after day 10 (FIG. 3D) in FR and FRW PKC-δ-Cre mice (dual ablation); graphs showing total food intake (g) during each day's feeding period (FIG. 3E) in FR (▴) and FRW (∘) no ablation (WT) groups and average food intake (g) across days 2-6 of the experiment (restriction began after day 1 feeding period) (FIG. 3F) in FR and FRW no ablation (WT) groups; graphs showing total food intake during each day's feeding period (FIG. 3G) in FR (▴) and FRW (∘) PKC-δ-Cre mice (dual ablation), and average food intake (g) across days 2-6 of the experiment (restriction began after day 1 feeding period) (FIG. 3H) in FR and FRW PKC-δ-Cre mice (dual ablation). Box plot averages based on days when more than one WT sample remained in the experiment. Dashed line at 20% weight loss indicates the point at which mice developed ABA and needed to be removed from the experiment to prevent death. Sudden changes in the line plots for WT mice (i.e., days 3-4) are due to the removal of a significant number of mice from the experiment; indicated by dashed section in line. All samples are females. *p<0.05, **p<0.01, ***p<0.001.

FIGS. 4A-4J are graphs showing mean daily total wheel revolutions for WT mice with no ablation (●; n=10) and PKC-δ-Cre mice (β; n=10) each day before (days −5 to −1) and during initial food restriction (FIG. 4A), and throughout light period (4 am-4 pm) (FIG. 4C), and throughout dark period (4 pm-4 am) (FIG. 4E) during baseline (food ad libitum, days −5 to −1) and during food restriction (days 1-10); and bar graphs showing average total revolutions before (days −5 to −1) and during initial food restriction (days 1-5 for light period and days 1-6 for dark period) for WT mice with no ablation (WT, n=10) and PKC-δ-Cre mice (Cre, n=10) of daily total (FIG. 4B, y-axis is mean daily total wheel revolutions), and throughout light period (FIG. 4D, y-axis is mean daily total wheel revolutions), and throughout dark period (FIG. 4F, y-axis is mean daily total wheel revolutions); and graphs showing mean total wheel revolutions during food anticipatory activity (FAA; 4 hours preceding presentation of food) each day of food restriction (FIG. 4G) and the average total revolutions during FAA at the initial food restriction period (days 1 to 5 or 6) (FIG. 4H, y-axis is mean daily total wheel revolutions) in WT mice with no ablation (●) and PKC-δ-Cre mice (∘); and graphs showing mean total wheel revolutions during feeding period each day of food restriction (FIG. 4I); and the average total revolutions during feeding period at the initial food restriction (days 1-5 or 6) (FIG. 4J, y-axis is mean daily total wheel revolutions) in WT mice with no ablation (●) and PKC-δ-Cre mice (∘). Box plot averages based on days when more than one WT sample remained in the experiment. Arrow indicates day in which food restriction (FR) was enforced. Sudden changes in the line plots for WT mice (i.e., days 3-4) are due to the removal of a significant number of mice from the experiment; indicated by dashed section in line. All samples are females. *p<0.05, **p<0.01, ***p<0.001.

FIGS. 5A-5I are graphs showing percent Fos⁺PKC-δ⁺ neurons (0%-40%) in the CeA (FIG. 5A) and ovBNST (FIG. 5B) brain regions, and total number of Fos⁺ neurons in the CeA (FIG. 5C) and ovBNST (FIG. 5D) brain regions of WT mice in the food restricted only (FR) condition (non-ABA; n=7), ABA resistant mice with food restriction and wheel (FRW non-ABA; n=3), and ABA susceptible mice with food restriction and wheel (FRW ABA; n=6); graphs showing percent Fos⁺PKC-δ⁺ neurons (0%-40%) (outcome variable) in the CeA (FIG. 5E) and ovBNST (FIG. 5F) brain regions versus food intake (percent body weight; predictor variable) across groups; graph showing food intake (percent body weight) in FR (non-ABA), FRW (non-ABA), and FRW (ABA) groups on day of removal from experiment (FIG. 5G); graph showing percent body weight loss on day of removal in FRW (non-ABA) and FRW (ABA) groups (FIG. 5H); graph showing total wheel activity (number of revolutions) on the two full days preceding removal in FRW (non-ABA) and FRW (ABA) groups (FIG. 5I). All samples are females. *p<0.05, **p<0.01, ***p<0.001.

FIGS. 6A-6C are graphs showing fluorescence intensity for each region where there are fluorescent axons and terminals using viral tracers simultaneously label PKC-δ+ neurons in the CeA and ovBNST, alternating with EYFP (FIG. 6A) and mCherry (FIG. 6B). FIG. 6C is a schematic diagram of the intra-circuit projections between the CeA and BNST PKC-δ+ neurons. No differences were seen between male and female mice. *p<0.05, **p<0.01, ***p<0.001.

FIGS. 7A-7D are a schematic diagram showing optogenetic activation of CeAPKC-δ neurons with chemogenetic silencing of ovBNSTPKC-δ neurons (FIG. 7A), and graph showing food intake (grams) in mice with or without optogenetic activation of CeAPKC-δ neurons with chemogenetic silencing of ovBNSTPKC-δ neurons as indicated (FIG. 7B); a schematic diagram showing optogenetic activation of ovBNSTPKC-δ neurons with chemogenetic silencing of CeAPKC-δ neurons (FIG. 7C), and graph showing food intake (grams) in mice with or without optogenetic activation of ovBNSTPKC-δ neurons with chemogenetic silencing of CeAPKC-δ neurons as indicated (FIG. 7D). In FIGS. 7B and 8D, from left to right: control (saline), chemogenetic silencing only, optogenetic activation only, simultaneous optogenetic activation and chemogenetic silencing. Data from male and female mice. p<0.05, **p<0.01, ***p<0.001.

FIGS. 8A-8J are graphs showing survival curve of WT mice with no ablation (FIG. 8A), average body weight loss (0% to −30%) over days of food restriction of WT mice with no ablation (FIG. 8B), and average percentage of body weight loss on day of removal from ABA experiment of WT mice with no ablation, either when the ABA criteria is reached (20% loss from baseline weight two days in a row) or after day 10 in male and female groups (FIG. 8C, y-axis is average percentage of body weight loss); graphs showing survival curve of PKC-δ-Cre mice with dual ablation (FIG. 8D), average percentage of body weight loss (0% to −30%) over days of food restriction of PKC-δ-Cre mice with dual ablation (FIG. 8E), and average percentage of body weight loss on day of removal from ABA experiment of PKC-δ-Cre mice with dual ablation, either when the ABA criteria is reached (20% loss from baseline weight two days in a row) or after day 10 in male and female groups (FIG. 8F, y-axis is average percentage of body weight loss). (WT p=0.004, male n=10, female n=10; PKC-δ-Cre p=0.93; male n=7 and female n=10). FIG. 8G is a graph showing the total food intake (g) during each day's feeding period; and FIG. 8H is a graph showing average food intake (g) across days 2-6 of experiment in male (▪) and female (∘) groups of WT mice with no ablation (FIG. 8H, y-axis is average food intake (g)). FIG. 8I is a graph showing the total food intake (g) during each day's feeding period; and FIG. 8J is a graph showing average food intake (g) across days 2-6 of experiment in male (▪) and female (∘) groups of PKC-δ-Cre mice with dual ablation (FIG. 8J, y-axis is average food intake (g)). Box plot averages based on days when more than one WT sample remained in the experiment. Arrow indicates day in which food restriction (FR) was enforced. Dashed line at 20% weight loss indicates the point at which mice have developed ABA and need to be removed from the experiment to prevent death. Sudden changes in the line plots for female WT mice (i.e., days 3-4) are due to the removal of a significant number of mice from the experiment; indicated by dashed section in line. *p<0.05, **p<0.01, ***p<0.001.

FIGS. 9A-9H are graphs showing number of total wheel revolutions per day during baseline before food restriction (food ad libitum, days −5 to −1) and during food restriction (days 1-10) (FIG. 9A), and average total wheel activity before and during initial food restriction (days 1-5) (FIG. 9B) in male (▪) and female (∘) groups of WT mice with no ablation; total wheel revolutions per day during baseline before food restriction (food ad libitum, days −5 to −1) and during food restriction (days 1-10) (FIG. 9C), and average total wheel activity before and during initial food restriction (days 1-5) (FIG. 9D) in male (▪) and female (∘) groups of PKC-δ-Cre mice with dual ablation; graphs showing average number of total wheel revolutions during light period (4 am-4 pm) across days of food restriction (FIG. 9E), and average total revolutions during initial food restriction (days 1-5) (FIG. 9F, y-axis is average number of total revolutions) in male (▪) and female (∘) groups of WT mice with no ablation; average number of total wheel revolutions during light period (4 am-4 pm) across days of food restriction (FIG. 9G), and average total revolutions during initial food restriction (days 1-5) (FIG. 9H, y-axis is average number of total revolutions) in male (▪) and female (∘) groups of PKC-δ-Cre mice with dual ablation. Box plot averages based on days when more than one WT sample remained in the experiment. Arrow indicates day in which food restriction (FR) was enforced. Sudden changes in the line plots for female WT mice (i.e., days 3-4) are due to the removal of a significant number of mice from the experiment; indicated by dashed section in line. *p<0.05, **p<0.01, ***p<0.001.

FIGS. 10A-10H are graphs showing number of total wheel revolutions per day during food restriction (days 1-10) (FIG. 10A, male groups (solid line) and female groups (dashed line)) and across days 1-5 of food restriction (FIG. 10B) for total wheel activity during food anticipatory activity (FAA, 4 hours preceding food) in male and female groups of WT mice with no ablation; graphs showing number of total wheel revolutions per day during food restriction (days 1-10) (FIG. 10C) and across days 1-5 of food restriction (FIG. 10D) for total wheel activity during food anticipatory activity (FAA, 4 hours preceding food) in male and female groups of PKC-δ-Cre mice with dual ablation; graphs showing number of total wheel revolutions per day during food restriction (days 1-10) (FIG. 10E) and across days 1-5 of food restriction (FIG. 10F) for total wheel activity during dark period (4 pm-4 am) in male and female groups of WT mice with no ablation; graphs showing number of total wheel revolutions per day during food restriction (days 1-10) (FIG. 10G) and across days 1-5 of food restriction (FIG. 10H) for total wheel activity during dark period (4 pm-4 am) in male and female groups of PKC-δ-Cre mice with dual ablation.

### DETAILED DESCRIPTION OF THE INVENTION

### I. Definitions

The terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. A subject can include a control subject or a test subject.

The term “dosage regime” refers to drug administration regarding formulation, route of administration, drug dose, dosing interval and treatment duration.

The term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered. The effect of the effective amount can be relative to a control. Such controls are known in the art and discussed herein, and can be, for example the condition of the subject prior to or in the absence of administration of the drug, or drug combination.

The term “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The term “pharmaceutically acceptable salt”, as used herein, refers to derivatives of the compounds defined herein, wherein the parent compound is modified by making acid or base salts thereof. Lists of suitable salts are found in Remington's Pharmaceutical Sciences, 20th ed., Lippincott Williams & Wilkins, Baltimore, MD, 2000, p. 704; and “Handbook of Pharmaceutical Salts: Properties, Selection, and Use,” P. Heinrich Stahl and Camille G. Wermuth, Eds., Wiley-VCH, Weinheim, 2002.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%.

The term “treating” or “preventing” a disease, disorder, or condition includes ameliorating at least one symptom of the disease or condition. Desirable effects of treatment include decreasing the rate of disease progression, ameliorating, or palliating the disease state, and remission or improved prognosis. For example, an individual is successfully “treated” if one or more symptoms associated with one or more eating disorders are mitigated or eliminated, including, but are not limited to, decreasing symptoms resulting from the disease, increasing the quality of life of those suffering from the disease, decreasing the dose of other medications required to treat the disease, delaying the progression of the disease, and/or prolonging survival of individuals.

The term “biodegradable”, generally refers to a material that will degrade or erode under physiologic conditions to smaller units or chemical species that are capable of being metabolized, eliminated, or excreted by the subject. The degradation time is a function of composition and morphology.

### II. Compositions

Compositions for treating or preventing one or more eating disorders including anorexia nervosa, bulimia, and related clinical syndromes in a subject in need thereof are described.

It has been established that simultaneous ablation of a specific subpopulation of neurons, marked by the expression of protein kinase C-delta (PKC-δ), in two nuclei of the central extended amygdala (EAc) rescues all three key phenotypes of the activity-based anorexia (ABA) animal model: extreme increases in wheel activity, decreases in food intake, and, consequently, life-threatening loss in body weight. Specifically, elimination of these neurons rescues the drastic reduction in body weight, insufficient food intake, and hyperactivity phenotypes, which are key characteristics in the progression of ABA development.

Previous work identified that neurons expressing protein kinase C-delta (PKC-δ) in two distinct nuclei of the central extended amygdala (EAc)—the central nucleus of the amygdala (CeA) and oval region of the bed nucleus of the stria terminalis (ovBNST)—suppress food intake when acutely activated (Cai, H., et al., Nat Neurosci, 2014. 17(9): p. 1240-8; Wang, Y., et al., Nat Commun, 2019. 10(1): p. 2769). The CeA^{PKC-δ} neurons are activated by satiety, visceral malaise nausea, and aversive taste sensation of bitter, while the ovBNST^{PKC-δ} neurons are activated by inflammatory signals related to sickness such as interleukin-1-beta (IL-1β), lipopolysaccharides (LPS), and tumor necrosis factor (TNF-α). Silencing of these neurons attenuates the anorexigenic effect caused by the respective signals.

Typically, the compositions include one or more inhibitors suitable for treating one or more eating disorders including anorexia nervosa, bulimia, and related clinical syndromes in a subject.

A. Inhibitors of PKC-δ-Expressing Neurons in the Central Nucleus of the Amygdala (CeA) and Oval Region of the Bed Nucleus of the Stria Terminalis (ovBNST)

Typically, the compositions include one or more inhibitors that can inhibit PKC-delta neurons in the central nucleus of the amygdala (CeA) and/or oval region of the bed nucleus of the stria terminalis (ovBNST).

In some embodiments, the inhibitor is DAMGO [D-Ala2, NMe-Phe4, Gly-ol5]-enkephalin, a highly selective peptide agonist for the u opioid receptor. Structure of DAMGO is shown below.

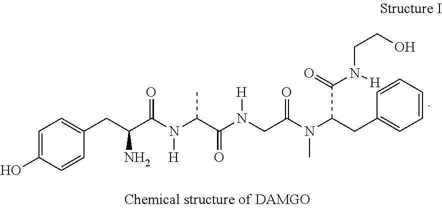 [SMILES: C[C@@H](NC(=O)[C@@H](N)Cc1ccc(O)cc1)C(=O)NCC(=O)N(C)[C@@H](Cc1ccccc1)C(=O)NCCO]

In other embodiments, the inhibitor is [Thr4,Gly7]-oxytocin (TGOT), a selective oxytocin receptor agonist. In some instances, the composition contains an effective amount of TGOT to activate one or more PKC-δ negative neurons downstream to the PKC-δ neurons.

In some embodiments, the inhibitor is corticotropin releasing factor (CRF). In some instances, the composition contains an effective amount of CRF to inhibit activities of one or more PKC-δ neurons in the central nucleus of the amygdala (CeA) and oval region of the bed nucleus of the stria terminalis (ovBNST).

In some embodiments, a first inhibitor inhibits or kills PKC-delta neurons in the CeA and a second inhibitor inhibits or kills PKC-delta neurons in the ovBNST. In other embodiments, the same inhibitor inhibits or kills PKC-delta neurons in both the central nucleus of the amygdala (CeA) and oval region of the bed nucleus of the stria terminalis (ovBNST). In further embodiments, the first inhibitor and the second inhibitor are in the same composition.

### B. Kits

The compositions can be packaged in kit. The kit can include a single dose or a plurality of doses of a composition including one or more inhibitors in a pharmaceutically acceptable carrier for shipping and storage and/or administration, and instructions for administering the compositions. Specifically, the instructions direct that an effective amount of the composition be administered to an individual with a particular disease/disorder as indicated. The composition can be formulated as described above with reference to a particular treatment method and can be packaged in any convenient manner. Components of the kit may be packaged individually and can be sterile.

### III. Methods of Selection of Therapeutic Agents

Methods of screening for suitable therapeutic agents to inhibit and/or kill PKC-delta neurons in the central nucleus of the amygdala (CeA) and/or in the oval region of the bed nucleus of the stria terminalis (ovBNST) are also described.

In some embodiments, one or more candidate drugs are administered to the desired regions of the brain and the activity of the neurons in or near the part of the brain is monitored. If the candidate drug inhibits and/or kills PKC-delta neurons in the central nucleus of the amygdala (CeA) and/or in the oval region of the bed nucleus of the stria terminalis (ovBNST), then the candidate drug is selected as a potential therapeutic agent for treating one or more eating disorders.

In other embodiments, the screening begins with in vitro assays. For example, brain slices with PKC-δ neurons are prepared and incubated with fluorescent calcium indicators. When the neurons are activated, intracellular calcium levels increase and the activated neurons show an increase in fluorescence. When the neurons are inhibited, their fluorescence decreases. Thus, potential drugs can be applied to screen for those drugs that can inhibit PKC-δ neurons or activate PKC-δ negative neurons. In some embodiments, the candidate drugs target upstream and/or downstream of PKC-δ neurons. Optionally, the candidate drugs target both upstream and downstream of PKC-δ neurons.

### IV. Methods of Treatment

Methods of treating or preventing one or more eating disorders including anorexia nervosa and bulimia, and related clinical syndromes in a subject in need thereof are described.

Methods can include electric or magnetic stimulation to reduce or inhibit the activity of CeA PKC-δ neurons and ovBNST PKC-δ neurons in the brain of a subject in need thereof. The methods provide an effective amount of electric stimulation to reduce or inhibit the activity of CeA PKC-δ neurons and ovBNST PKC-δ neurons in the brain of the subject, preferably, in an amount effective to increase or excite the activity of the brain regions downstream of CeA PKC-δ neurons and ovBNST PKC-δ neurons such as medial part of CeA and ventral lateral part of BNST. In other embodiments, electric stimulations are administered to kill one or more CeA PKC-δ neurons and ovBNST PKC-δ neurons.

Methods can also include administering one or more pharmacological inhibitors suitable for treating one or more eating disorders including anorexia nervosa, bulimia, and related clinical syndromes in a subject. The methods provide an effective amount of one or more pharmacological inhibitors to reduce or inhibit the activity of CeA PKC-δ neurons and ovBNST PKC-δ neurons in the brain of the subject, preferably, in an amount effective to increase or excite the activity of the brain regions downstream of CeA PKC-δ neurons and ovBNST PKC-δ neurons such as medial part of CeA and ventral lateral part of BNST.

In further embodiments, transcranial magnetic stimulation (TMS) is used to stimulate parts of the brain to treat an eating disorder such as anorexia, to reduce or inhibit the activity of CeA PKC-δ neurons and ovBNST PKC-δ neurons in the brain of the subject, preferably, in an amount effective to increase or excite the activity of the brain regions downstream of CeA PKC-δ neurons and ovBNST PKC-δ neurons such as medial part of CeA and ventral lateral part of BNST. In one embodiment, repetitive transcranial magnetic stimulation (rTMS) is used (Muratore A F, et al., Int J Eat Disord. 2021 November; 54 (11): 2031-2036).

In some embodiments, methods target brain regions upstream of PKC-δ neurons. For example, the upstream neurons that excite PKC-δ neurons can be inhibited for therapy, while upstream neurons that inhibit PKC-δ neurons can be excited for therapy. Many upstream regions of PKC-δ neurons previously identified; for example, insular cortex, piriform cortex, and entorhinal cortex send excitatory inputs to PKC-δ neurons, while the arcuate nucleus sends inhibitory inputs to PKC-δ neurons (Cai, H., et al., Nat Neurosci, 2014. 17(9): p. 1240-8; Wang, Y., et al., Nat Commun, 2019. 10(1): p. 2769).

A. Methods of Inhibiting CeA PKC-δ Neurons and ovBNST PKC-δ Neurons

In some embodiments, one or more pharmacological drugs are administered to inhibit or kill one or more CeA PKC-δ neurons and ovBNST PKC-δ neurons. Preferably, the active agents are administered via local administration, i.e., directly in the brain tissue, by direct infusion or intracranial convection-enhanced delivery (CED).

Alternately, they can be administered intravenously, or intra-arterially via catheter into an artery that serves the region of the brain to be treated.

### B. Methods of Deep Brain Stimulation

Deep brain stimulation (DBS) is a neurosurgical procedure that involves the use of electrodes that are implanted into specific targets in the brain to deliver programmed electric stimulation (A. M. Lozano, et al. Nat Rev Neurol, 15(2019), pp. 148-160; and L. Pycroft, et al. Brain Neurosci Adv, 2 (2018), Article 2398212818816017; Hsu T I et al., World Neurosurgery, 2022, doi.org/10.1016/j.wneu.2022.09.114).

In some embodiments, DBS selectively targets the central amygdala, ovBNST, or their downstream regions, such as the medial part of central amygdala and ventral lateral part of BNST, in a dosage effective to treat an eating disorder such as anorexia. In some embodiments, DBS selectively targets the SCC (ventral to the corpus callosum) and the NAcc (rostral to the hypothalamus) in an amount effective to increase BMI of patients, for example one with refractory AN. In some embodiments, the mean maxima for current ranges about 1.5-5.5 mA, inclusive, preferably about 3.64 mA; voltage about 2.5-8.0 V, inclusive, preferably about 4.21 V; electric application time about 60-350 milliseconds, inclusive, preferably about 115.87 milliseconds; and frequency stimulation settings about 130-204 Hz, inclusive, preferably about 114.6 Hz. In preferred embodiments, the DBS is administered in an amount effective to increase BMI up to about 5%, 10%, 15%, 20%, 25%, 30%, 35%, 40%, 45%, 50%, 55%, 60%, 65%, 70%, inclusive, to a healthy range of between about 18.5 and about 24.9.

### C. Conditions to be Treated

### 1. Eating Disorders

Eating disorders are serious and often fatal illnesses that are associated with severe disturbances in people's eating behaviors and related thoughts and emotions. Eating disorders include preoccupation with food, body weight, and shape. Common eating disorders include anorexia nervosa, bulimia nervosa, and binge-eating disorder.

Anorexia nervosa (AN), a serious psychiatric disorder with unknown etiology, is associated with restricted eating, intense fear of graining weight, excessive exercise, and emotional conditions such as anxiety and depression. People with anorexia nervosa avoid food, severely restrict food, or eat very small quantities of only certain foods. They also may weigh themselves repeatedly. Even when dangerously underweight, they may perceive themselves as overweight. There are two subtypes of anorexia nervosa: a restrictive subtype and a binge-purge subtype. People with the restrictive subtype of anorexia nervosa severely limit the amount and type of food they consume. People with the binge-purge subtype of anorexia nervosa also greatly restrict the amount and type of food they consume. In addition, they may have binge-eating and purging episodes—eating large amounts of food in a short time followed by vomiting or using laxatives or diuretics to get rid of what was consumed. Symptoms of anorexia nervosa including but not limited to thinning of the bones (osteopenia or osteoporosis), mild anemia and muscle wasting and weakness, brittle hair and nails, dry and yellowish skin, growth of fine hair all over the body (lanugo), severe constipation, low blood pressure, slowed breathing and pulse, damage to the structure and function of the heart, brain damage, multiorgan failure, drop in internal body temperature, causing a person to feel cold all the time, lethargy, sluggishness, or feeling tired all the time, and infertility.

In some embodiments, the patient has chronic anorexia. The terms “severe and enduring anorexia nervosa,” “chronic anorexia nervosa” and “chronic anorexia” are used herein interchangeably and are defined as having symptoms of anorexia nervosa persisting for a long period of time despite treatment. In some embodiments, severe and enduring anorexia nervosa, or chronic anorexia nervosa, in adults refers to having symptoms persisting for at least about 3 years despite treatment involving at least two modalities, such as different forms of therapy.

Various criteria may also be relied on to identify such individuals and may include reliance on the DSM (Diagnostic & Statistical Manual) which uses Body Mass Index (BMI) as an index for eating disorder severity and classifies anorexia nervosa and BN based on BMI as follows: Mild BMI >17 kg/m²; Moderate BMI 16-16.99 kg/m²; Severe BMI 15-15.99 kg/m²; and Extreme BMI <15 kg/m².

Accordingly, in one embodiment the individual with a history consistent with an eating disorder such as anorexia nervosa has a BMI from about 15 kg/m² to about 25 kg/m²; in another embodiment the BMI is from about 16 kg/m² to about 20 kg/m²; in another embodiment the BMI is from about 18 kg/m² to about 19 kg/m²; in another embodiment the BMI is from about 15 kg/m² to about 18 kg/m²; and in another embodiment the BMI is from about 15 kg/m² to about 17 kg/m²; and in another embodiment the BMI is from about 15 kg/m² to about 16 kg/m². In some embodiments, the subject has a BMI of less than about 15 kg/m² and a medical history consistent with anorexia. A medical history consistent with anorexia can include, but not limited to, continuing to be plagued by disturbing compulsions to exercise excessively, eat in a restricted manner, and have intense negative self-thoughts and anxieties about food, body image, or exercising. In some embodiments, the subject is diagnosed with or suspected of having anorexia nervosa and failed to show significant improvement with usual care, including refeeding, family involvement in refeeding, and personal therapy for about 6 months.

Bulimia nervosa is a condition where people have recurrent and frequent episodes of eating unusually large amounts of food and feeling a lack of control over these episodes. This binge-eating is followed by behavior that compensates for the overeating such as forced vomiting, excessive use of laxatives or diuretics, fasting, excessive exercise, or a combination of these behaviors. People with bulimia nervosa may be slightly underweight, normal weight, or overweight. Symptoms of bulimia nervosa include chronically inflamed and sore throat, swollen salivary glands in the neck and jaw area, worn tooth enamel and increasingly sensitive and decaying teeth as a result of exposure to stomach acid, acid reflux disorder and other gastrointestinal problems, intestinal distress and irritation from laxative abuse, severe dehydration from purging of fluids, electrolyte imbalance (too low or too high levels of sodium, calcium, potassium, and other minerals) which can lead to stroke or heart attack.

Methods are suitable for patients diagnosed with an eating disorder. Thus, in some embodiments, the methods include the step of diagnosis of a patient suspected of having an eating disorder. Step of diagnosis may include physical exam, for example, measuring height and weight; checking vital signs, such as heart rate, blood pressure and temperature; checking skin and nails for problems; listening to heart and lungs; and examining abdomen; lab tests, for example, a complete blood count (CBC) and more-specialized blood tests to check electrolytes and protein as well as functioning of your liver, kidney and thyroid; or a urinalysis; psychological evaluation, for example, a doctor or mental health professional will likely ask about thoughts, feelings and eating habits, or by completing psychological self-assessment questionnaires; and other studies, for example, X-rays to check your bone density, check for stress fractures or broken bones, or check for pneumonia or heart problems, electrocardiograms to look for heart irregularities.

Methods also include the step of selecting a patient suitable for treatment by the steps described above. In some embodiments, the subject is currently undergoing psychotherapy for eating disorders include anorexia nervosa, bulimia nervosa, and binge-eating disorder.

### D. Combination Therapies

In some embodiments, patients are also subject to conventional therapy to treat eating disorders include anorexia nervosa, bulimia nervosa, and binge-eating disorder. Exemplary conventional therapies include psychotherapy, antidepressants, and nutritional counseling.

The disclosed methods of treating or preventing one or more eating disorders can be further understood through the following numbered paragraphs.
1. A method of treating or preventing one or more eating disorders, comprising administering to a subject in need thereof an effective amount of electric stimulation to reduce or inhibit the activity of CeA PKC-δ neurons and ovBNST PKC-δ neurons in the brain of the subject.
2. A method of treating or preventing one or more eating disorders, comprising administering to a subject in need thereof an effective amount of electric stimulation to increase or excite the activity of the brain regions downstream of CeA PKC-δ neurons and ovBNST PKC-δ neurons of the subject.
3. The method of paragraph 2, wherein the brain regions downstream of CeA PKC-δ neurons and ovBNST PKC-δ neurons are selected from the group consisting of medial part of CeA, ventral lateral part of BNST, and combinations thereof.
4. A method of treating or preventing one or more eating disorders, comprising administering to a subject in need thereof an effective amount of electric stimulation to increase or excite the activity of one or more brain regions upstream of CeA PKC-δ neurons and ovBNST PKC-δ neurons of the subject.
5. The method of paragraph 4, wherein the one or more brain regions upstream of CeA PKC-δ neurons and ovBNST PKC-δ neurons send inhibitory inputs to PKC-δ neurons.
6. The method of paragraph 5, wherein the one or more brain regions upstream of CeA PKC-δ neurons and ovBNST PKC-δ neurons are arcuate nucleus.
7. A method of treating or preventing one or more eating disorders, comprising administering to a subject in need thereof an effective amount of electric stimulation to reduce or inhibit the activity of one or more brain regions upstream of CeA PKC-δ neurons and ovBNST PKC-δ neurons of the subject.
8. The method of paragraph 7, wherein the one or more brain regions upstream of CeA PKC-δ neurons and ovBNST PKC-δ neurons send excitatory inputs to PKC-δ neurons.
9. The method of paragraph 8, wherein the one or more brain regions upstream of CeA PKC-δ neurons and ovBNST PKC-δ neurons is selected from the group consisting of the insular cortex, piriform cortex, and entorhinal cortex, and combinations thereof.
10. A method of treating or preventing one or more eating disorders, comprising administering to a subject in need thereof an effective amount of a first pharmacological drug to reduce or inhibit the activity of CeA PKC-δ neurons and ovBNST PKC-δ neurons in the brain of the subject.
11. The method of paragraph 10, wherein the first pharmacological drug can inhibit the activity of CeA PKC-δ neurons or ovBNST PKC-δ neurons, or both.
12. The method of paragraph 10, wherein the first pharmacological drug can inhibit the activity of CeA PKC-δ neurons, and wherein the method further comprises administering a second pharmacological drug can inhibit the activity of ovBNST PKC-δ neurons, wherein the first pharmacological drug and the second pharmacological drug are administered simultaneously or sequentially.
13. The method of any one of paragraphs 10-12, wherein the first pharmacological drug is administered locally to the brain, optionally, wherein the pharmacological drug is administered locally to the CeA PKC-δ neurons and/or ovBNST PKC-δ neurons in the brain.
14. A method of treating or preventing one or more eating disorders, comprising administering to a subject in need thereof an effective amount of a first pharmacological drug to increase or excite the activity of one or more brain regions downstream of CeA PKC-δ neurons and ovBNST PKC-δ neurons of the subject.
15. The method of paragraph 14, wherein the one or more brain regions downstream of CeA PKC-δ neurons and ovBNST PKC-δ neurons are selected from the group consisting of the medial part of CeA, ventral lateral part of BNST, and a combination thereof.
16. A method of treating or preventing one or more eating disorders, comprising administering to a subject in need thereof an effective amount of a pharmacological drug to increase or excite the activity of one or more brain regions upstream of CeA PKC-δ neurons and ovBNST PKC-δ neurons of the subject.
17. The method of paragraph 16, wherein the one or more brain regions upstream of CeA PKC-δ neurons and ovBNST PKC-δ neurons send inhibitory inputs to PKC-δ neurons.
18. The method of paragraph 17, wherein the one or more brain regions upstream of CeA PKC-δ neurons and ovBNST PKC-δ neurons is in the arcuate nucleus.
19. A method of treating or preventing one or more eating disorders, comprising administering to a subject in need thereof an effective amount of a pharmacological drug to reduce or inhibit the activity of one or more brain regions upstream of CeA PKC-δ neurons and ovBNST PKC-δ neurons of the subject.
20. The method of paragraph 19, wherein the one or more brain regions upstream of CeA PKC-δ neurons and ovBNST PKC-δ neurons send excitatory inputs to PKC-δ neurons.
21. The method of paragraph 20, wherein the one or more brain regions upstream of CeA PKC-δ neurons and ovBNST PKC-δ neurons is selected from the group consisting of the insular cortex, piriform cortex, and entorhinal cortex, and combinations thereof.
22. The method of any one of paragraphs 1-21, wherein the one or more eating disorders is selected from the group consisting of anorexia nervosa, bulimia nervosa, and binge-eating disorders.
23. The method of any one of paragraphs 1-22, wherein the one or more eating disorders is anorexia nervosa.
24. The method of any one of paragraphs 1-22, wherein the one or more eating disorders is bulimia nervosa.

The present invention will be further understood by reference to the following non-limiting examples.

### EXAMPLES

### Example 1: Preventing Activity-Based Anorexia with a Subpopulation of Neurons in Two Nuclei of the Central Extended Amygdala

### Material and Methods

Mice. PKC-δ-Cre C57BL/6 mice (Haubensak, W., et al., Nature, 2010. 468 (7321): p. 270-6) were crossed with wildtype (WT) C57BL/6 mice from the Charles River Laboratory to get PKC-δ-Cre or WT in this study; the same mouse line as was used in previous studies (Cai, H., et al., Nat Neurosci, 2014. 17(9): p. 1240-8; Wang, Y., et al., Nat Commun, 2019. 10(1): p. 2769). The genotype of offspring generated and used from these mice were determined by PCR of genomic tail DNA. Stereotaxic survival surgery was performed when mice were 2-3 months old. All mice were housed on a 12-hours light (4 am)/dark (4 pm) cycle, with ad libitum access to water and rodent chow, except for during the ABA experiment food restriction and food intake tests.

All animal care and experimental procedures were in accordance with ethical regulations, conducted according to the National Institutes of Health guidelines for animal research, and approved by the Institutional Animal Care and Use Committee (IACUC) at the University of Arizona.

Virus and tracer. For Cre-dependent ablation, rAAV2-FLEX-taCasp3-Tevp, a virus generated by Dr. Nirao Shah's lab, was used. For Cre-dependent anterograde tracing, rAAV2-EF1a-DIO-EYFP and rAAV5-hSyn-DIO-mCherry, generated by Dr. Karl Deisseroth's lab and by Dr. Bryan Roth's lab, respectively, were used. For optogenetic activation, we used rAAV2-EF1a-DIO-ChR2-EYFP generated by Dr. Karl Deisseroth's lab. For chemogenetic silencing, rAAV5-hSyn-DIO-hM4Di-mCherry generated by Dr. Bryan Roth's lab was used. These viral constructs were deposited and packaged into viral vectors either at the University of North Carolina (UNC) Viral Vector Core or Addgene at a titer of 4-6×10¹² genome copies per ml. Upon arrival to our lab, the virus stocks were aliquoted and stored at −80° C. until used.

Stereotaxic survival surgery. All mouse surgeries were performed using aseptic techniques with a stereotaxic frame (Model 1900 Stereotaxic Alignment System, Kopf Instruments), as previously described (Wang, Y., et al., Nat Commun, 2019. 10(1): p. 2769). Injection coordinates (in mm) relative to midline, bregma, and skull surface at bregma were followed as (x, y, z): ovBNST (÷1.13, +0.3, −4.1) and CeA (±2.85, −1.40, −4.7). Viruses were microinfused through a pulled-glass micropipette with 20-50 μm tip outer diameter connected with a Nanoliter Injector (Nanoliter 2010, World Precision Instruments) at a rate of 10 units per minute (1 unit=4 nl). After each injection, the micropipette was left in place for 5 min to allow for diffusion of the liquid, followed by withdrawal of 5 units at the same rate, 0.3 mm in the +y direction to remove any unwanted spread of virus. Injection volume for the caspase virus was 55 units (220 nl) and for the anterograde tracer was 40 units (160 nl) per injection location. The caspase virus was injected bilaterally and the virus for tracing experiments was injected unilaterally. For the optogenetic tests, optical ferrule fibers were implanted bilaterally ˜0.5 mm above the injection coordinates. After ferrule fiber implantation, dental cement (C&B Metabond) was used to secure the fiber to the skull. For the 3 days after surgery, mice were injected intraperitoneally with ketoprofen (5 mg/kg). Mice were allowed three weeks after virus injection surgery for recovery and viral expression before used for behavioral experiments or euthanized for tracing analysis.

Activity-Based Anorexia (ABA). Stereotaxic surgery was performed on all mice undergoing the ABA protocol (Welch, A. C., W. R. Katzka, and S. C. Dulawa, J Vis Exp, 2018(135)). After 2-3 weeks for recovery and gene expression, mice were individually housed, and a wheel was added to their respective home cages. Control mice without a wheel were individually housed at the same time. Food and water were given ad libitum for the next 5-7 days, while establishing a baseline of wheel activity, body weight, and food eaten. Total food and body weight of each experimental mouse was measured daily during the hour before start of light off/active period. Mice were habituated to the experimenter handling during this time, as well. MED Associates Inc low-profile Wireless Running Wheels (ENV-047) with the respective Wheel Manager Software was used for continually recording running data. After establishing the baseline and acclimating the mice to the new environmental condition for 5-7 days, followed by food restriction. On the first day, mice were given food for 5 hours, starting at the beginning of the dark cycle. On the second day, mice were given food for 4 hours, still starting at the onset of the dark cycle period. From the third day until the end of the study, mice were given food for 3 hours, again starting at the beginning of the dark cycle.

Each day, mice—and their respective wheels—were transferred from their home cage with bedding to an empty cage, for the sake of measuring the food consumed most accurately. More than enough pre-weighed regular chow (NIH-31, Zeigler Bros, Inc) was provided in the empty cage (˜10 g total). Water was given ad libitum, both during the feeding period and during the food restriction period. After the feeding period, mice were weighed and transferred back into their original home cages with the wheel if they had one. Red light was used in the dark room to prevent disruption to the light cycle and circadian rhythm. In another room, total food left was then measured for each sample, and calculated from what was given to determine amount eaten. The empty cages were cleaned and used again for the rest of the experiment.

For ethical purposes, mice were removed from the experiment when body weight loss exceeded 20% of their respective baseline weight two days in a row, when measured before given food. If the mouse's body weight remained under 20% after the feeding period on the first day of that measurement, it was also removed. Mice were monitored on the day after reaching 20% and were removed from the experiment if they appeared to be in critical condition or reached 20% loss before group measurement time, in order to prevent unexpected deaths. Therefore, since mice were removed at various times on their second day of being below 20% weight loss, the final day of running wheel activity data was not included in analysis for all mice who developed ABA. Removal from the experiment meant mice were given ad libitum food immediately, while the wheel was taken out and a cardboard house was placed in the home cage.

Immunohistochemistry and histology. Immunostaining and histology analyses were performed in order to check (1) the level of ablation of PKC-δ⁺ neurons after the ABA experiments, (2) c-Fos expression in PKC-δ⁺ neurons, and (3) virus expression for the tracing experiments. All mice were deeply anesthetized with ketamine/xylazine and perfused with 20 ml PBS followed by 20 ml of 4% paraformaldehyde (PFA) in PBS. Mice were perfused 90 minutes after presentation food in the c-Fos experiment. The brains were then extracted, post-fixed in 4% PFA overnight at 4° C., rinsed with PBS, and then sectioned with a vibratome (Leica, VT1000S). Brains from mice that underwent the ABA experiment were sliced at 100 μm thickness, while brains from the viral tracing were sliced at 150 μm thickness. The brain slices with the viral tracing were mounted on glass slides and imaged. The brain slices from ABA mice were stained with antibodies to tag all neurons (NeuN) and neurons expressing PKC-δ. Brain tissue slices were stained with primary antibody at 4° C. overnight, in a blocking solution containing 5% donkey serum and 0.5% Triton X-100. After three rounds of 5-10-minute washes in PBS with 0.1% Triton X-100 solution, the tissues were incubated in secondary antibodies in the PBS-0.1% Triton X-100 at room temperature for 1-2 hours. Tissue slices were then washed for three times for 5-10 minutes in PBS before being mounted on glass slides. Vectashield mounting medium was added before placing coverslips on top. Imaging was done using a ZEISS AxioZoom v16 Fluorescent Microscope with Apotome 2 Structured Illumination Module for optical sectioning.

Primary antibodies used were rabbit anti-PKC-δ (Abcam, ab182126, 1:1000), guinea pig anti-NeuN (Fisher/Sigma, ABN90MI, 1:1000), and guinea pig c-Fos antibody (SYSY, 266 308, 1:5000). Secondary antibodies used were Alexa Fluor 488 donkey anti-rabbit IgG (Jackson Immuno Research Inc. 711-545-152, 1:500) and Alexa Fluor 594 donkey anti-guinea pig IgG (Jackson Immuno Research Inc. 50-194-3535, 1:500).

Food intake with chemogenetic silencing and optogenetic activation. After three days of habituation for at least 20 min each day, mice were food-deprived, with water provided ad libitum, one day before test. Mice were briefly anaesthetized with isoflurane and coupled with optic fibers and Clozapine-N-oxide (CNO) IP injection (Enzo life science-Biomol, BML-NS105-0005, freshly dissolved in 0.9% NaCl saline to a concentration of 1 mg/ml) at 5 mg/kg. Saline was injected as vehicle control. After at least 25 min of recovery, optogenetic activation was performed as previously described (Cai, H., et al., Nat Neurosci, 2014. 17(9): p. 1240-8; Wang, Y., et al., Nat Commun, 2019. 10(1): p. 2769). Food intake was then measured in a 20 min feeding session. The light was delivered by a blue laser (Shanghai DreamLaser: 473 nm, 50 mW) just after the mice were introduced into the testing cage. To be consistent with previous experiments (Cai, H., et al., Nat Neurosci, 2014. 17(9): p. 1240-8; Wang, Y., et al., Nat Commun, 2019. 10(1): p. 2769), 15 Hz, 10 ms light pulses were used to activate ovBNSTPKC-6 neurons, while 5 Hz, 10 ms light pulses were used to activate CeA^{PKC-δ} neurons.

Quantification and statistical analysis. All WT and PKC-δ-Cre mice that were injected with the taCasp3 virus (see Virus and tracer section for details) were perfused, and brains were extracted for analysis of ovBNST^{PKC-δ} and CeA^{PKC-δ} cell population (see Immunohistochemistry and histology section for details). Data from WT mice that underwent food restricted (FR) and food restricted with wheel (FRW) conditions for the C-Fos experiment were collected and analyzed in the same way. For the ovBNST, 3-4 brains sections that included anterior, middle, and posterior ovBNST regions were analyzed and averaged per animal. Similarly, for the CeA, 6-8 brain sections that included anterior, middle, and posterior CeA were analyzed and averaged per animal. The number of PKC-δ⁺ cells per area of interest (ovBNST and CeA) were quantified using the multi-point tool/cell counter plug-in in FIJI/ImageJ. Samples with partial ablation were included, as this ablation technique is not always absolutely complete, or to the same degree in the exact same parts of the region targeted (ovBNST and/or CeA). To determine and calculate the percentage of PKC-δ⁺ cells expressing C-fos in the ovBNST and CeA, the cells simultaneously expressing PKC-δ and c-Fos were counted with the channels tool, divided by the total PKC-δ⁺ cells in the same region, and multiplied by 100.

As much of the brains as possible from the PKC-δ-Cre mice injected with tracing virus were sliced with the vibratome and imaged with the AxioZoom (see previous sections for details). The FIJI/ImageJ software was used to measure the level of fluorescence. For each region, the measurements were averaged across all slices in which they appeared. The Mouse Brain In Stereotaxic Coordinates (Franklin, K. B. J. and G. Paxinos, Third ed. 2007: Academic Press is an imprint of Elsevier) was used for reference to identify location of fluorescence. The regions were grouped and designated as follows: bed nucleus of the stria terminalis (BNST, oval and ventral-lateral; plates 29-31), extended BNST (ST, plates 32-36), extended amygdala and medial central amygdaloid nucleus (EAC/M, CeM; plates 37-40), central amygdaloid nucleus (CeA, CeM, CeL, CeC; plates 41-46), parasubthalamic nucleus (PSTh; plates 46-51), ventrolateral medial reticular formation (vlmRt, plates 55-62), lateral parabrachial nucleus (LPB, plates 76-79).

Data were analyzed with RStudio or GraphPad Prism Software. Line plot error bars represent mean±s.e.m. Box plots show interquartile range (IQR), ranging from 25^{th} to 75^{th} percentile, with the bar in the box representing 50^{th} percentile (median). Top/bottom whiskers are the largest/smallest value within 1.5 times IQR above/below the 75^{th} and 25^{th} percentile, respectively. Outside values beyond either end of the box and error bars are greater than 1.5 times the IQR. Unpaired t-test with Bonferroni adjustment method was used to compare two groups with one variable. One-way ANOVA was used to compare three or more groups with one variable, and two-way ANOVA for groups with more than one variable; both with Tukey HSD post-hoc analysis. Survival analysis plots display the Kaplan-Meier estimate of time-to-event (i.e., development of ABA) with right censoring method to account for subjects that had not developed ABA by the end time point (day 10). Log-rank test was used to determine if there was a statistically significant difference in survival curves between groups. Shading around curves represent the 95% confidence intervals for the point estimates. A p-value less than 0.05 was considered significant.

### Results

CeA^{PKC-δ} and ovBNST^{PKC-δ} Neurons Regulate ABA Development.

To investigate how CeA or ovBNST PKC-δ+ neurons might play a role in the development of AN, we assessed how mice respond to the ABA paradigm when this subpopulation of neurons was ablated. We first focused only on female mice because human AN is observed and diagnosed disproportionally more in females (Culbert, K. M., C. L. Sisk, and K. L. Klump, Clin Ther, 2021. 43 (1): p. 95-111).

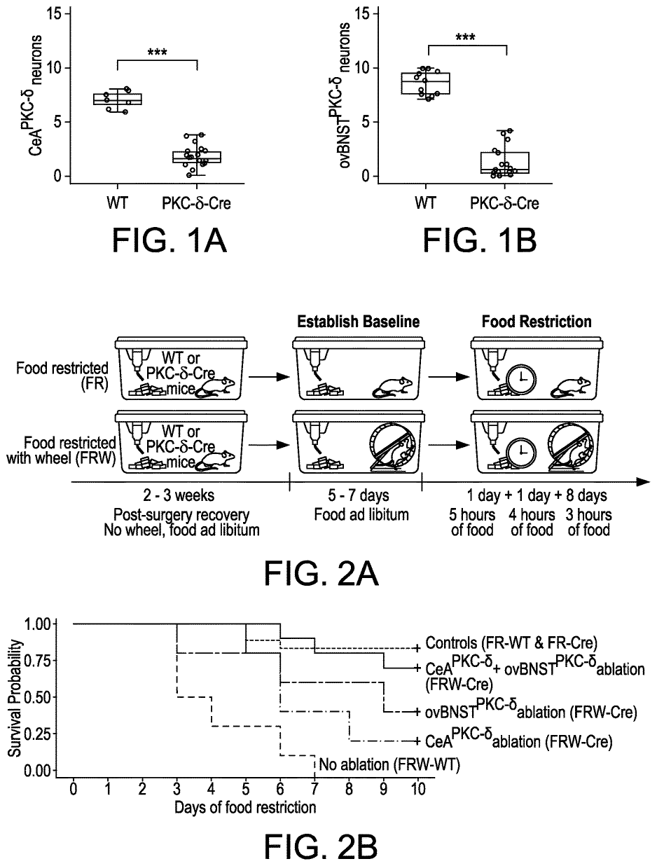

To ablate the PKC-δ neurons, a Cre-dependent adeno-associated virus (AAV) expressing caspase (AAV2-FLEX-taCasp3) bilaterally was stereotaxically injected into either the CeA or ovBNST—or into both simultaneously—of PKC-δ-Cre mice (histology data not shown). Wildtype (WT) mice were injected with the same virus for use as controls. Following behavior experiments, ablation success was evaluated and confirmed with immunohistochemistry and cell quantification of the labelled neurons (FIGS. 1A-1B). Comparison of body weight from before virus injection to three weeks after demonstrates ablation itself did not significantly impact body weight. Interestingly, even though acute silencing of CeA and ovBNST PKC-δ neurons increases food intake, chronic ablation in either or both nuclei did not increase total daily food intake, as determined before any food restriction was enforced. Additionally, there was no consistent significant difference in total daily wheel activity or body mass in mice with PKC-δ neuron ablation compared to WT mice before any food restriction. These baseline data indicate there is no significant change in body weight, food intake, or wheel activity when the mice are in equivalent conditions with ad libitum access to food after EAc^{PKC-δ} neuron ablation.

After three weeks post-surgery for recovery time and gene expression, the ABA assay was applied (Welch, A. C., W. R. Katzka, and S. C. Dulawa, Assessing Activity-based Anorexia in Mice. J Vis Exp, 2018 (135)) (FIG. 2A). All WT mice in the food restricted with wheel group (FRW) developed ABA within the 10 days (FIG. 2B). Food restricted only (FR) included all mice that did not have a wheel; they functioned as a control group because they are usually able to survive the new limited feeding schedule (Francois, M. and L. M. Zeltser, Curr Psychiatry Rep, 2022. 24(1): p. 71-76). While ablation of PKC-δ neurons in either the CeA or ovBNST offered some level of protection compared to WT mice, simultaneous ablation in both regions was required for consistent prevention of ABA (FIG. 2B). Mice with dual ablation showed a strong significant difference in survival compared to WT FRW, and no significant difference compared to ABA controls (FR) (FIG. 2B). These data suggest bilateral, dual ablation of PKC-δ neurons in two nuclei of the EAc—the CeA and the ovBNST—have a compounding effect to regulate development of ABA.

Dual Ablation of CeA^{PKC-δ} and ovBNST^{PKC-δ} Neurons Rescues the Reduced Body Weight and Food Intake in ABA.

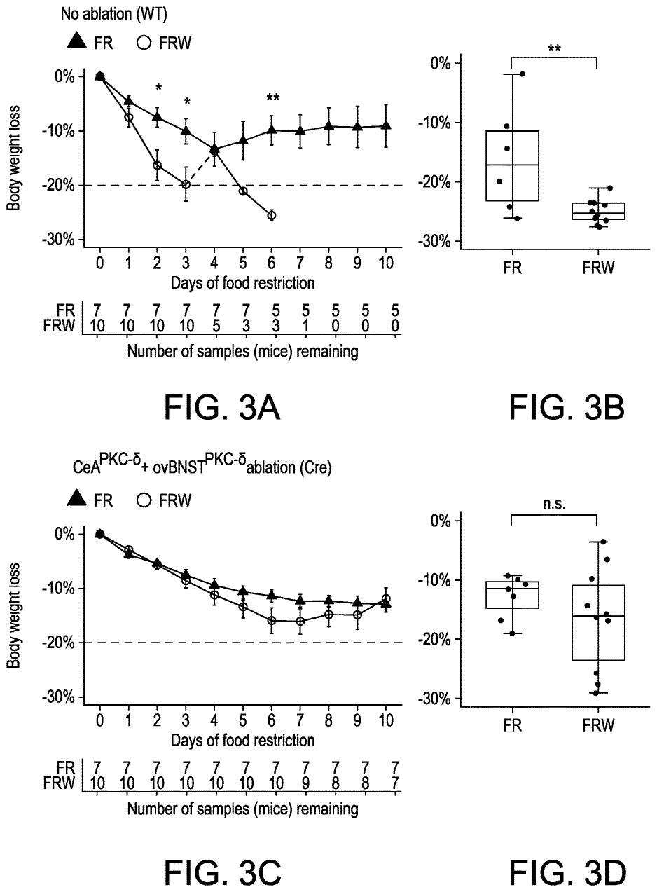

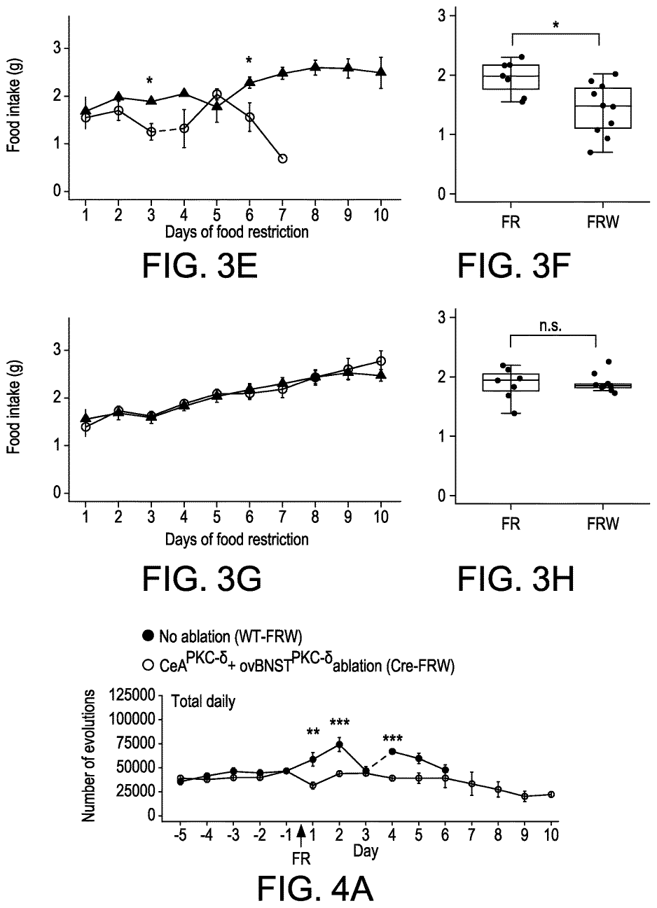

In order to determine how the mice with dual EAc^{PKC-δ} ablation are able to survive the ABA conditions, we first assessed body weight change across days of the experiment, as well as corresponding food intake. Consistent with previous studies (Beneke, W. M., S. E. Schulte, and J. G. vander Tuig, An analysis of excessive running in the development of activity anorexia. Physiol Behav, 1995. 58 (3): p. 451-7), the baseline data showed that running wheel itself for mice with ad libitum food did not significantly impact weight loss. FR mice, however, did decrease their body weight, but not typically to the life-threatening point requiring removal from the experiment (FIGS. 2B and 3A-3D). Therefore, the FR group functioned as a control in this study. As expected, there were significant differences in body weight loss between FRW WT mice (no ablation) and their respective FR controls (FIGS. 3A-3B). However, in contrast, FRW and FR PKC-δ-Cre mice (dual ablation) followed almost identical trends across all 10 days (FIGS. 3C-3D). Correspondingly, food intake was clearly disrupted and irregular with WT FRW mice, decreasing across days as the mice lost weight (FIGS. 3E-3F), while PKC-δ-Cre FRW food intake very closely matched that of the respective PKC-δ-Cre FR only control group (FIGS. 3G-3H). Individual sample data plots comparing FRW WT mice (no ablation) to FRW PKC-δ-Cre mice (dual ablation) further demonstrate mice with dual ablation as being less susceptible to developing ABA. Specifically, their body weights tended to level out at a survivable point (less than 20% loss from baseline), in contrast to the WT mice (no ablation), who exhibited extreme decrease in body weight during the first few days of food restriction, to the point of requiring removal from the experiment in order to prevent death. Additionally, mice with dual ablation tended to gradually increase their food intake across days of limited food time exposure, and especially more consistently than compared to the WT mice. In summary, these data show how dual ablation of EAc^{PKC-δ} neurons causes the mice to behave more similarly to their respective controls—food restricted (FR) only—to become more “resilient”, with adaptive increases in food intake and eventual weight stabilization (Beeler, J. A. and N. S. Burghardt, Bio Protoc, 2021. 11(9): p. e4009; and Beeler, J. A., et al., Biol Psychiatry, 2021. 90(12): p. 829-842).

Dual Ablation of CeA^{PKC-δ} and ovBNST^{PKC-δ} Neurons Rescues the Light Period Hyperactivity in ABA.

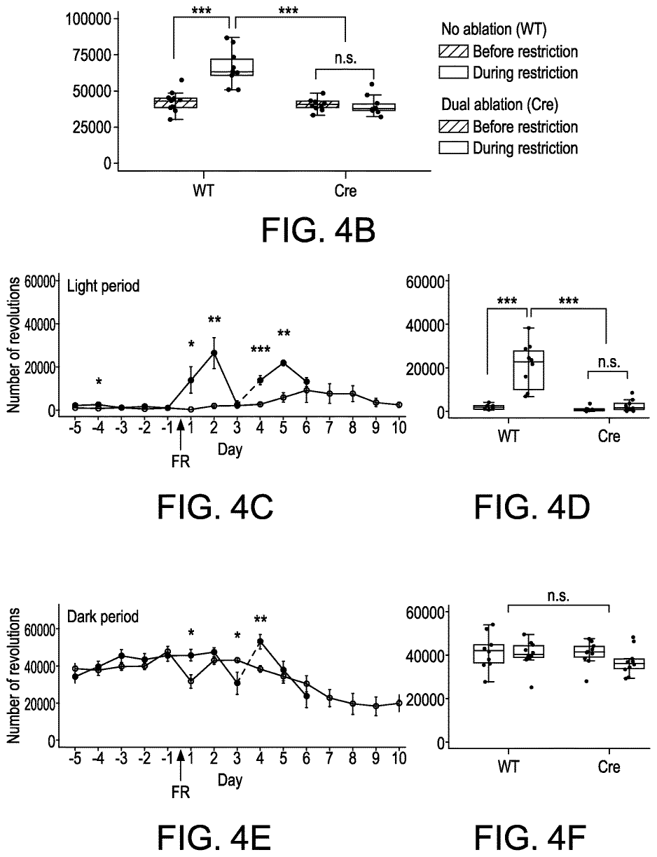

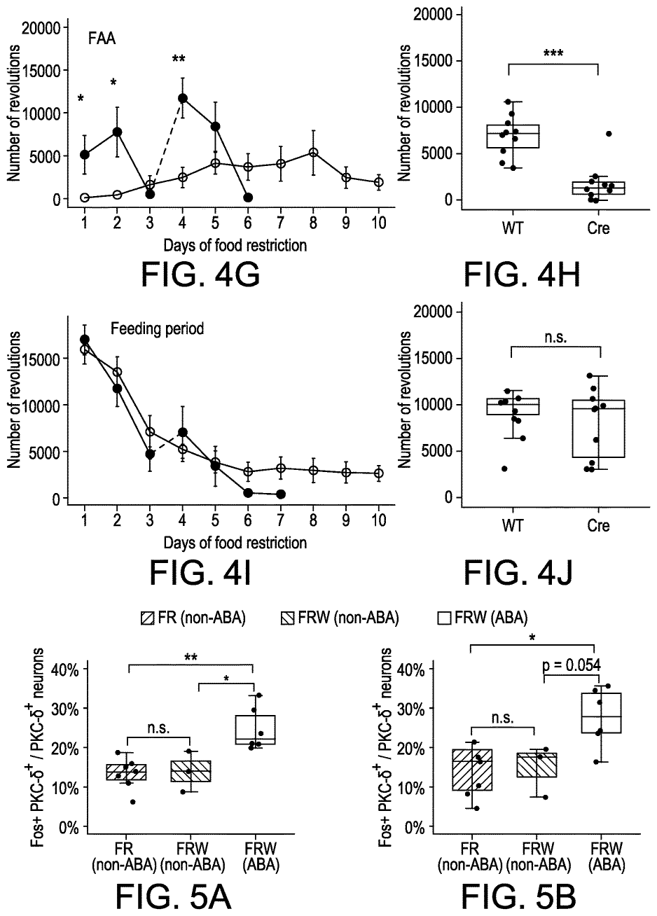

Another crucial element to development of ABA is increased hyperactivity (Pierce, W. D., et al., Behav Anal, 1994. 17 (1): p. 7-23; Chowdhury, T. G., Y. W. Chen, and C. Aoki, J Vis Exp, 2015 (105): p. e52927). Daily running wheel activity in WT (no ablation) mice was not significantly different compared to PKC-δ-Cre mice (dual ablation) before food restriction begins (FIGS. 4A-4B). However, upon the onset of food restriction (indicated by the arrow in FIG. 4A), FRW mice with no ablation significantly increased their daily wheel activity, while FRW mice with dual ablation were not significantly affected. Mice with no ablation demonstrated hyperactivity across days of food restriction until removal of wheel was required to prevent death, whereas mice with dual ablation tended to show more steady wheel activity, suggesting adaptation to food restriction parameters.

To investigate details of the wheel activity, the recorded data were divided into different time frames for each day: light period, dark period, food anticipatory period (FAA, defined as four hours preceding food presentation in the light period; Mistlberger, R. E., Neurosci Biobehav Rev, 1994. 18 (2): p. 171-95), and feeding period (FIGS. 4C-4J). When comparing the population mean of mice with dual ablation (PKC-δ-Cre) to mice with no ablation (WT), mice with dual ablation do not demonstrate extreme hyperactivity during the light period, which includes FAA (FIGS. 4C-4D and 4G-4H). Additionally, the dark period wheel activity for mice with dual ablation is comparable to that before food restriction, and even decreases with days of food restriction (day 2 vs. day 10, p=0.0002), suggesting a level of arousal adaptation that corresponds with the new feeding schedule (FIGS. 4E-4F). Consistent with previous results, time series data show that WT mice had a significant disruption and abnormality with strong wheel activity one or two days before they had to be removed from the experiment. In contrast, dual ablation mice showed a consistent pattern of day/night wheel activity, with moderate activity during the dark period (“awake” time) and minimal activity during the light period (“rest/sleep” time). No significant difference in the wheel activity during feeding time was observed (FIGS. 4I-4J).

Together, these data demonstrate mice with dual ablation survive ABA conditions primarily by not increasing wheel activity upon the introduction of the new feeding schedule, as well as by gradually increasing food intake across days.

### ABA Causes More EAc PKC-δ Neurons to be Activated in Response to Food.

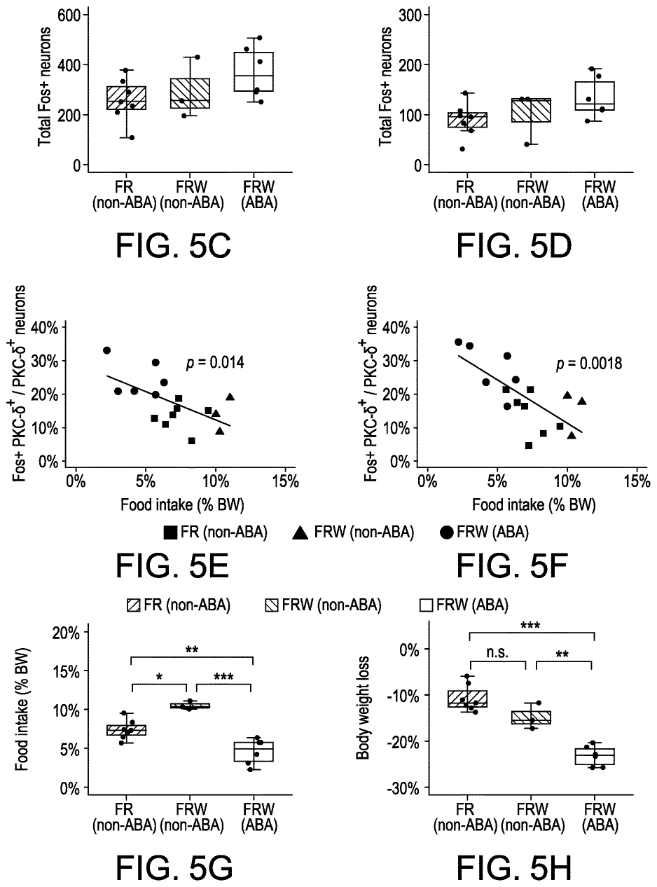

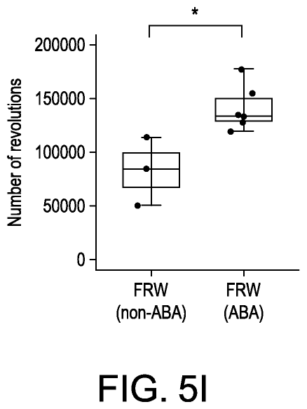

The results thus far indicate that PKC-δ neurons in both the CeA and ovBNST contribute to the development of ABA. Previous studies demonstrated that activation of CeA^{PKC-δ} or OvBNST^{PKC-δ} neurons suppresses food intake. Correspondingly, it was hypothesized that FRW mice have an increased activity of CeA^{PKC-δ} and ovBNST^{PKC-δ} neurons after ABA development. To investigate the involvement of the PKC-δ neuron activity in the four different brain regions (bilateral CeA and bilateral ovBNST) in mice developing ABA, c-Fos expression in FRW WT mice and their respective controls, FR only mice, were monitored. On the day in which FRW samples reached the criteria for developing ABA, they were perfused 90 minutes after presentation of food. FR samples, which do not develop ABA or reach that life-threatening body weight standard, were similarly collected on a day before the end of the 10-day experiment, also 90 minutes after presentation of food. Double immunostaining for c-Fos⁺ and PKC-δ⁺ neurons revealed that FRW ABA mice had significant increases in number of both CeA^{PKC-δ} and ovBNSTPKC-δ neurons expressing Fos compared to FR non-ABA mice (FIGS. 5A-5B). Both the right and left sides of the CeA and ovBNST show increased Fos expression in PKC-δ neurons, suggesting a bilateral importance. The total number of Fos-expressing neurons in these nuclei was not significantly different (FIGS. 5C-5D). Additionally, simple linear regression analysis indicates a significant negative correlation between food intake and c-Fos⁺PKC-δ⁺ neurons across groups (FIGS. 5E-5F), which aligns with the significantly less food intake in FRW ABA mice on day of removal from the experiment compared FR to non-ABA mice (FIG. 5G). Interestingly, there were a few FRW samples in this cohort that did not develop ABA within the 10-day experiment but were still collected for analysis purposes. ABA resistance and susceptibility is clear when comparing the body weight loss and wheel revolutions during days of food restriction for mice in the FRW condition (FIGS. 5H-5I). These ABA resistant mice (FRW non-ABA) show comparable levels of Fos expression in PKC-δ⁺ neurons to the FR non-ABA control mice, while significantly different than FRW ABA mice. The results here are consistent with the previously discovered function of EAc^{PKC-δ} neurons in suppressing food intake when activated and further support the involvement of CeA^{PKC-δ} and ovBNSTPKC-δ neurons in regulating the development of ABA.

CeA^{PKC-δ} and ovBNST^{PKC-δ} Neurons Function in Combination

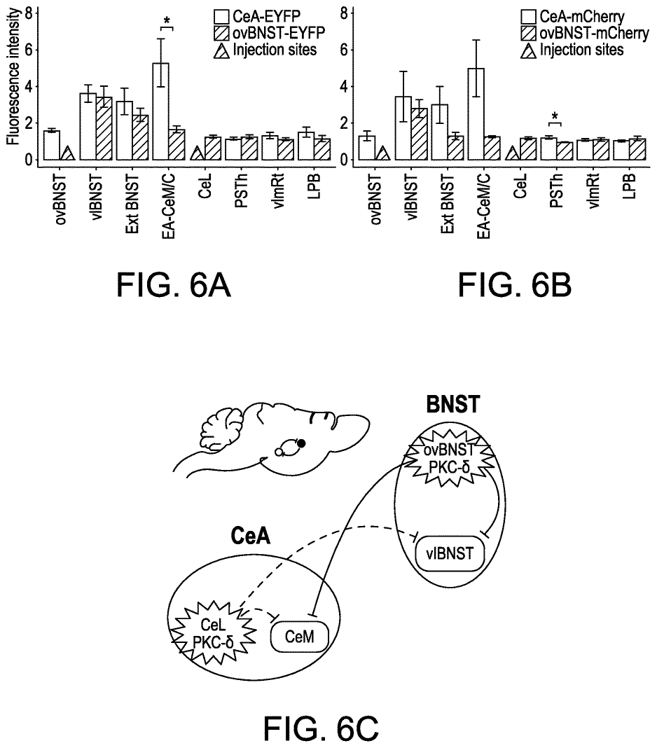

To determine how CeA^{PKC-δ} and ovBNST^{PKC-δ} neurons might exert their function at the circuit level, downstream projections from the specific PKC-δ neurons were examined in each of these discrete regions simultaneously using Cre-dependent viral tracing techniques. EYFP and mCherry were alternated to account for innate differences in fluorescence and it was found that fluorescence was largely at the same locations and with similar intensity for both the CeA and ovBNST (FIGS. 6A-6B). Note that the ovBNST and CeA have significantly increased fluorescence at those respective regions due to the cell bodies. Green and red fluorescence are graphed separately to account for innate differences in fluorescence, as EYFP is typically stronger than mCherry. CeA^{PKC-δ} neurons displayed their strongest projections, in order, at the medial central amygdala (CeM), extended BNST region, and ventrolateral BNST (vIBNST). ovBNST^{PRC-δ} neurons projections are similar, but with vlBNST being the strongest, followed by extended BNST and CeM. Both CeA^{PKC-δ} and ovBNST^{PKC-δ} neurons showed minor terminal fluorescence at the parasubthamalic nucleus (PSTh), ventrolateral medial reticular formation (vlmRt), and lateral parabrachial nucleus (LPB). Overall, this data demonstrates how GABAergic CeA^{PKC-δ} and ovBNST^{PKC-δ} neurons make similar contributions to intra-circuit inhibitory connections within the EAc (FIG. 6C), as well as long-range projections in multiple brain regions, suggesting that removal of one would have some disinhibitory effect, but not as great as if both were removed.

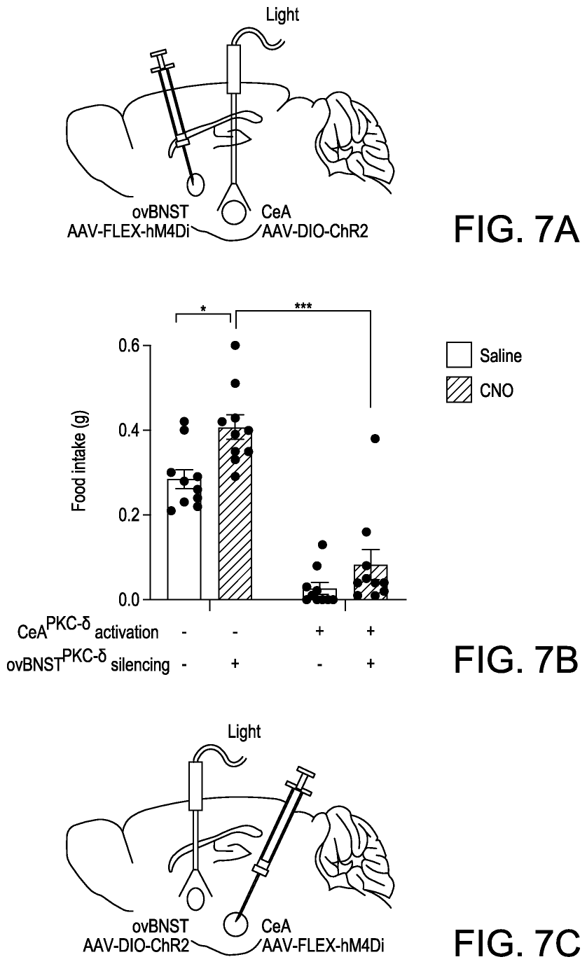

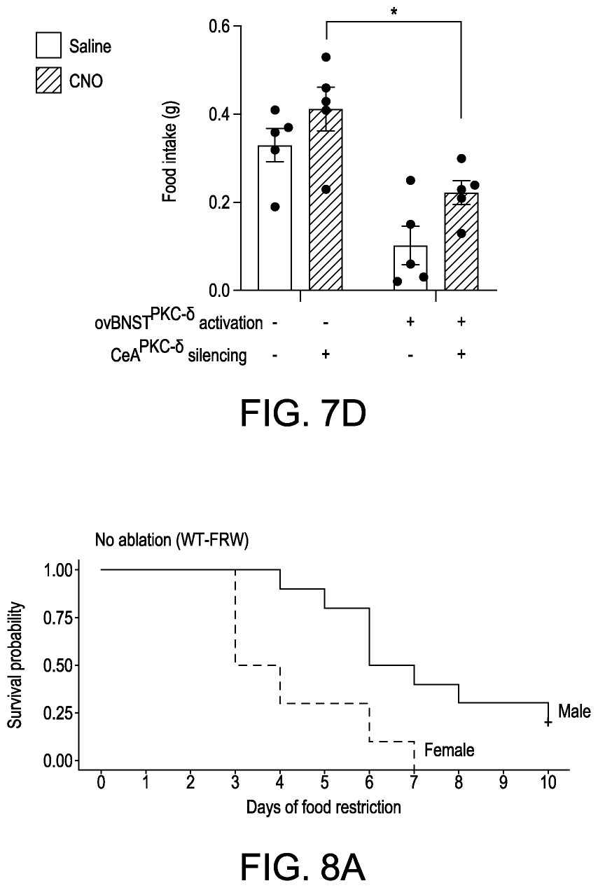

Previous results showed that optogenetic activation of the CeA^{PKC-δ} neurons or ovBNST^{PKC-δ} neurons suppresses food intake, while chemogenetic silencing of these neurons can block the anorexia induced by the corresponding anorexigenic signals that these neurons mediate. Thus, it was tested whether bilateral silencing of the PKC-δ neurons in one of the two nuclei would prevent the feeding suppression caused by activation of the other nuclei. AAV-DIO-ChR2-EYFP was stereotaxically injected in one of the nuclei, as well as AAV-DIO-hM4Di-mCherry in the other nuclei; both bilaterally (FIGS. 7A and 7C). Consistent with the previous study, chemogenetic inhibition of ovBNST^{PKC-δ} neurons increased food intake compared to saline (FIG. 7B), while inhibiting CeA^{PKC-δ} neurons had a non-significant trend of increase in food intake (FIG. 7D). When PKC-δ neurons in one of the nuclei were optogenetically activated, silencing of the PKC-δ neurons in the other nuclei caused a trend of increase in food intake, yet still significantly lower than no activation control. These results suggest that activation of either CeA^{PKC-δ} neurons or ovBNST^{PKC-δ} neurons alone is sufficient to suppress food intake, even when the other is silenced. This observation is consistent with the results from the current study that single ablation only had a mild attenuation on ABA while dual ablation can prevent ABA.

Dual Ablation of CeA^{PKC-δ} and ovBNST^{PKC-δ} Neurons Minimizes Sexual Divergence Seen in ABA.

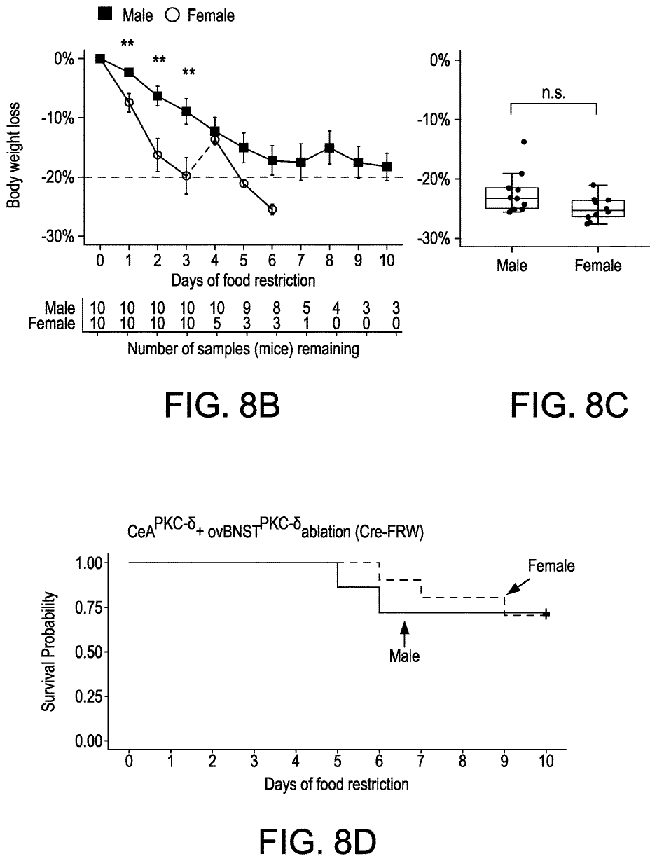

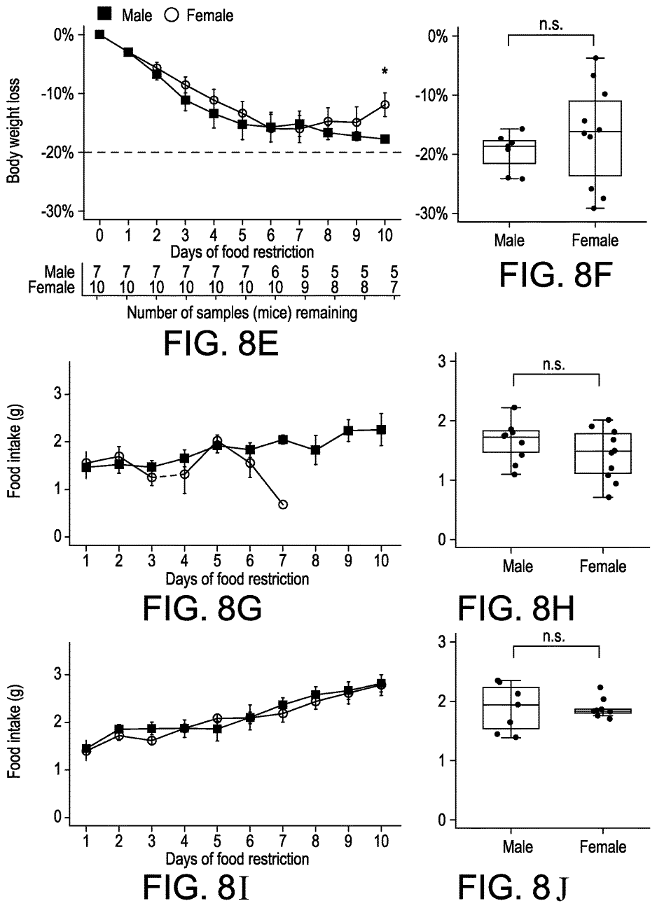

Previous literature showed that behavior and survival in the ABA paradigm can vary depending on sex (Kurnik-Łucka, M., Skowron, K., Gil, K., In Search for Perfection: An Activity-Based Rodent Model of Anorexia. Animal Models of Eating Disorders. Second ed. Neuromethods, ed. N. M. Avena. Vol. 161. 2021, New York. NY: Humana; Achamrah, N., et al., Physiol Behav, 2017. 170: p. 1-5). The WT mice did indeed demonstrate sexual divergence: the females consistently developed ABA quicker and lost weight more drastically than males (FIGS. 8A-8C). However, in contrast to these WT mice without ablation, PKC-δ-Cre mice with dual ablation did not demonstrate sexual divergence in the same way. Survival analysis indicates no significant difference in probability of developing ABA, while the trend of decrease in body weight was similar between the sexes (FIGS. 8D-8F). In addition, food intake did not significantly differ between sexes in each group (FIGS. 8G-8J).

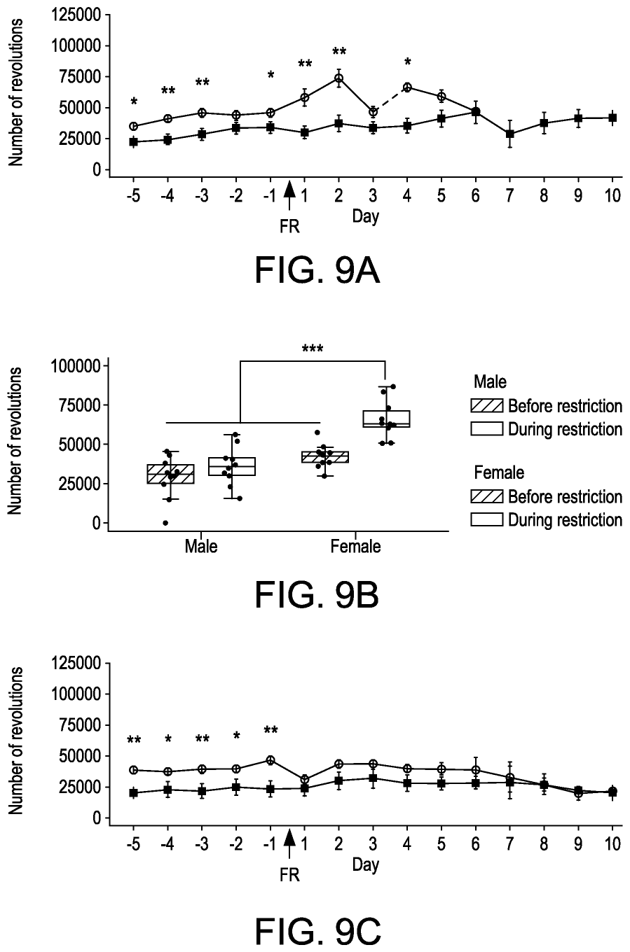

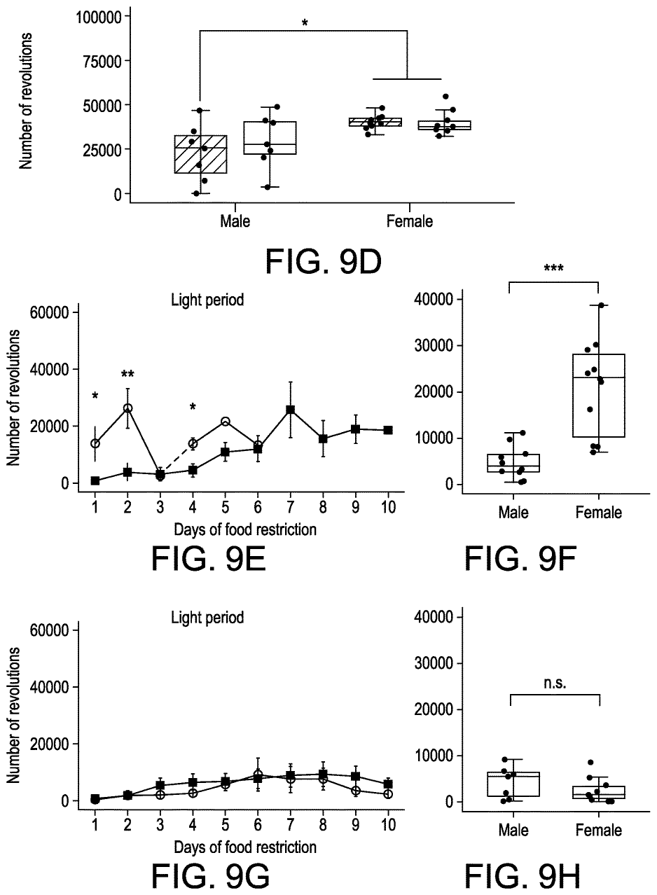

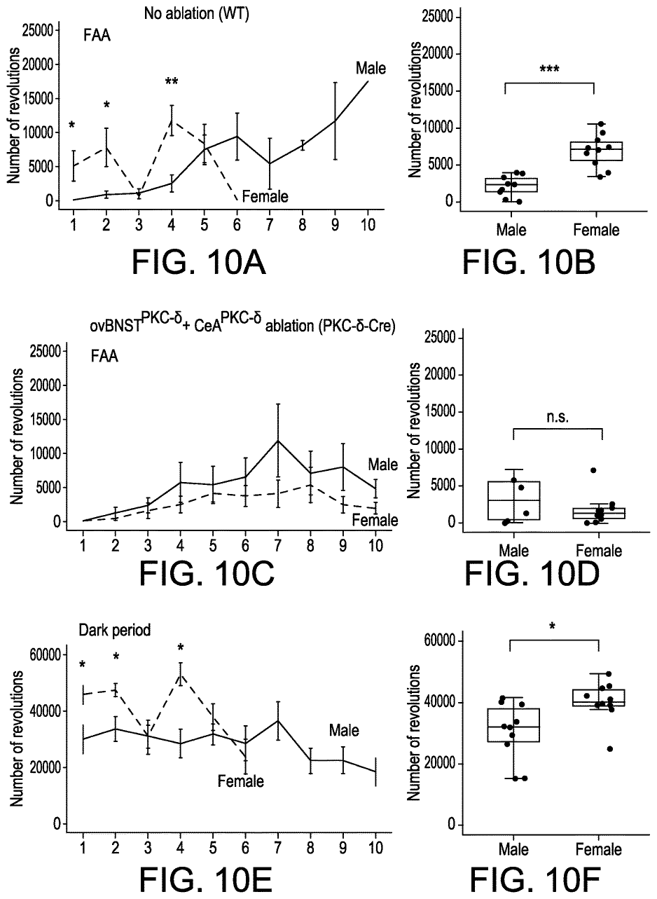

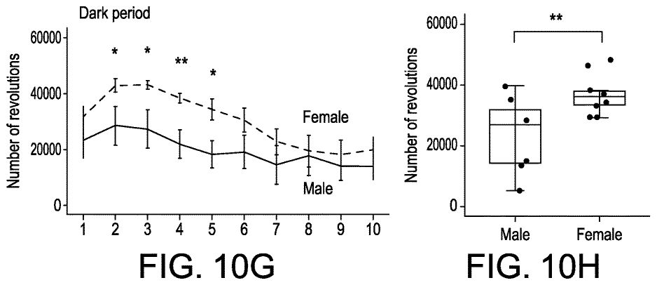

While food intake did not significantly differ between sexes in each group, variance is seen in the wheel activity (FIGS. 9A-9D). Consistent with previous studies looking at voluntary wheel activity by mice, there was a slight increase in daily wheel activity in females compared to males before food restriction started (for both groups) (Bartling, B., et al., Exp Gerontol, 2017. 87(Pt B): p. 139-147; Manzanares, G., G. Brito-da-Silva, and P. G. Gandra, Braz J Med Biol Res, 2018. 52(1): p. e7830). However, while the male and female mice with no ablation increased their differences in activity upon food restriction, the difference was minimized upon food restriction in mice with dual ablation. Accordingly, the wheel activity during the light period was significantly different between male and female mice with no ablation, but not with dual ablation mice (FIGS. 9E-9H). FAA, specifically, was also significantly different between sexes for WT mice (FIGS. 10A-10D). While dark period wheel activity was increased in females of both experimental groups compared to the respective males for the first 5 days of food restriction, the activity for both males and females with dual ablation decreased and plateaued to become insignificantly different for the latter half of food restriction days (FIGS. 10E-10H). Additionally, there was no difference in the wheel activity for the sexes of either group during the feeding period. Overall, decreased wheel activity during the light period indicates decreased susceptibility to development of ABA, which is seen with a portion of the male WT mice, as well as with majority of both male and female PKC-δ-Cre mice with dual ablation. Therefore, these data suggest EAc^{PKC-δ} neurons impact factors related to arousal and circadian rhythm changes induced by ABA that is typically different between sexes when the neurons are present.

The instant study shows how this particular subpopulation of neurons in the CeA and ovBNST regulates the development AN using the current best animal model of AN, activity-based anorexia (ABA), in which rodents develop self-starvation and hyperactivity tendencies when exposed to a restricted feeding schedule in combination with ad libitum access to a running wheel, to the point of death if not removed in time (Routtenberg, A. and A. W. Kuznesof, J Comp Physiol Psychol, 1967. 64(3): p. 414-21; Francois, M. and L. M. Zeltser, Curr Psychiatry Rep, 2022. 24(1): p. 71-76; Zhang, J. and S. C. Dulawa, Front Psychiatry, 2021. 12: p. 711181).

Here, it has been shown that bilateral, simultaneous ablation of CeA^{PKC-δ} and ovBNSTPKC-δ neurons prevents mice from developing ABA. Specifically, elimination of these neurons rescues the drastic reduction in body weight, insufficient food intake, and hyperactivity phenotypes, which are key characteristics in the progression of ABA development (Gutierrez, E., Int J Eat Disord, 2013. 46(4): p. 289-301; Klenotich, S. J. and S. C. Dulawa, Methods Mol Biol, 2012. 829: p. 377-93; Schalla, M. A. and A. Stengel, Front Nutr, 2019. 6: p. 69). Ablation of PKC-δ neurons in only one of the nuclei, however, was not sufficient to prevent ABA. Our data also demonstrate an increased number of activated CeA^{PKC-δ} and ovBNST^{PKC-δ} neurons in mice after ABA. Consistent with these results, it was observed that activation of PKC-δ neurons in one nucleus continues to suppress food intake even when PKC-δ neurons in the other nuclei are silenced. Additionally, it was further noted that ablation of these neurons minimized the difference in susceptibility to develop ABA between male and female mice. Together, our study demonstrates that neurons in the amygdala play a critical role in anorexia development.

### DISCUSSION

Anorexia nervosa (AN) is a psychiatric condition that also involves disruptions in eating behavior and energy homeostasis, making it an inherently complicated disorder. Consequently, the etiology of AN remains ambiguous. Although functional alterations of brain regions associated with AN have been observed in human neuroimaging studies (Bulik, C. M., et al., Nat Neurosci, 2022. 25 (5): p. 543-554; Kaye, W. H., et al., Trends Neurosci, 2013. 36 (2): p. 110-20; Ross, R. A., Y. Mandelblat-Cerf, and A. M. Verstegen, Harv Rev Psychiatry, 2016. 24 (6): p. 416-436), details of neural mechanisms that might cause AN are still being uncovered. Studies exploring these underlying neural processes often use the activity-based anorexia (ABA) animal model, which demonstrates three major characteristics leading to the development of anorexia: life-threatening decrease in body weight, reduced food intake, and increased running wheel activity overall or during the FAA period (Francois, M. and L. M. Zeltser, Curr Psychiatry Rep, 2022. 24 (1): p. 71-76; Zhang, J. and S. C. Dulawa, Front Psychiatry, 2021. 12: p. 711181; Schalla, M. A. and A. Stengel, Front Nutr, 2019. 6: p. 69; Ross, R. A., Y. Mandelblat-Cerf, and A. M. Verstegen, Harv Rev Psychiatry, 2016. 24 (6): p. 416-436). The increased running wheel activity, in particular, has been established as a robust predictor for the susceptibility of ABA (Chowdhury, T. G., Y. W. Chen, and C. Aoki, J Vis Exp, 2015 (105): p. e52927).

It has long been known that AN is comorbid with emotional conditions and, therefore, that development of the disorder may be attributed to the neural circuits that control emotions, especially those in the amygdala regions (Murray, S. B., et al., Neurosci Biobehav Rev, 2018. 95: p. 383-395; Hardaway, J. A., et al., Genes Brain Behav, 2015. 14 (1): p. 85-97). Consistent with this theory, neuroimaging studies have suggested that the function of amygdala or amygdala associated mesolimbic brain regions are altered in patients with AN (Bulik, C. M., et al., Nat Neurosci, 2022. 25 (5): p. 543-554; Kaye, W. H., et al., Trends Neurosci, 2013. 36 (2): p. 110-20; Ross, R. A., Y. Mandelblat-Cerf, and A. M. Verstegen, Harv Rev Psychiatry, 2016. 24 (6): p. 416-436). However, whether neurons in amygdala regulate ABA was not known. In fact, only recently have neurons in the amygdala, especially the CeA and BNST regions, been demonstrated to regulate eating behavior and eating suppression in anorexigenic conditions (Petrovich, G. D., et al., J Neurosci, 2009. 29 (48): p. 15205-12; Jennings, J. H., et al., Science, 2013. 341 (6153): p. 1517-21; Douglass, A. M., et al., Nat Neurosci, 2017. 20 (10): p. 1384-1394; Kim, J., et al., Neuron, 2017. 93 (6): p. 1464-1479 e5; Hardaway, J. A., et al., Neuron, 2019. 102 (5): p. 1037-1052 e7; Ip, C. K., et al., Cell Metab, 2019. 30 (1): p. 111-128 e6). Among these neurons, CeA^{PKC-δ} neurons are preferentially activated by anorexigenic signals such as satiety, visceral malaise and nausea, and bitter taste, but not by LPS-induced sickness. On the other hand, the ovBNST^{PKC-δ} neurons are preferentially activated by anorexia signals related to inflammation or sickness, such as IL-1β, LPS, and TNF-α, but not by the CCK satiation signal. Chemogenetic silencing of these PKC-δ neurons blocks the anorexigenic effect induced by the corresponding signals. Here, we demonstrate that single ablation of CeA^{PKC-δ} neurons or ovBNST^{PKC-δ} neurons has only a mild effect in attenuating ABA, while dual ablation of the PKC-δ neurons simultaneously in these two nuclei prevents ABA development. Notably, all key phenotypes are rescued—life-threatening body weight loss, insufficient food intake, overall running wheel hyperactivity, and increased FAA—to a level indistinguishable to their respective controls (FR only or access to wheel with food ad libitum). These results clearly demonstrate that neurons in the amygdala play a critical role in ABA development. Since these PKC-δ neurons regulate emotional behaviors or have wide interactions with the neurons that regulate emotions (Griessner, J., et al., Mol Psychiatry, 2021. 26 (2): p. 534-544; Haubensak, W., et al., Nature, 2010. 468 (7321): p. 270-6), a future direction of how emotions such as depression and anxiety contribute to AN development, or are affected by AN, is warranted.

While acute manipulations of the CeA^{PKC-δ} or ovBNST^{PKC-δ} neurons affect food intake, significant changes in food intake or body weight were not observed after ablation of these neurons during the baseline period with food ad libitum, suggesting a possible compensation mechanism after ablation. However, the results show that the activity of these PKC-δ neurons is significantly increased in response to food after ABA development (FIGS. 5A-5I), which is consistent with their anorexigenic effect when activated, as well as the fact that ablation of these neuron can restore the food intake and body weight in the ABA paradigm. Importantly, these data also clearly demonstrate that ablation of these neurons rescues the wheel hyperactivity phenotype seen in ABA. Therefore, the current study offers a novel role of these neurons in regulating both energy intake and expenditure in the context of stressful conditions that contributes to ABA development.

The neural tracing results demonstrate that PKC-δ neurons in CeA and ovBNST have similar downstream targets, which aligns with previous work indicating similar anatomical connections of the CeA and BNST neurons (Ye, J. and P. Veinante, Brain Struct Funct, 2019. 224 (3): p. 1067-1095). In combination with this evidence, the requirement of simultaneously ablating PKC-δ neurons in two brain regions to sufficiently block all key characteristics observed in ABA supports the notion of neurons in each of these distinct regions as having different functions, yet in a way that is integrated and coordinated. Furthermore, this necessity for dual ablation suggests that ABA development involves contribution from a combination of multiple anorexigenic factors rather than a single factor. Similarly, the notion that phasic fear is associated with the CeA, while sustained fear and anxiety are identified with the BNST (Davis, M., et al., Neuropsychopharmacology, 2010. 35 (1): p. 105-35; Kim, S. Y., et al., Nature, 2013. 496 (7444): p. 219-23)—two types of stress responses that could be independently relevant in ABA and AN—further supports the significance of both regions and the heterogenous aspect of such disorders. Given that these neurons are characterized as being GABAergic (Cassell, M. D., L. J. Freedman, and C. Shi, Ann N Y Acad Sci, 1999. 877: p. 217-41), ablation would lead to elimination of inhibitory projections, thus, disinhibition of downstream targets. Removing only one of the inhibitions may not be sufficient for complete disinhibition. Another possibility for why single ablation does not have the same effect is that they may innervate different subpopulations, even if they project to similar brain regions, but future experiments are needed for confirmation. Consistent with this concept of both nuclei being necessary, we demonstrate that food intake is still suppressed when the PKC-δ neurons in one nucleus is activated while the other is silenced (FIGS. 7A-7D). Additional work should go into identifying the downstream disinhibited neurons in order to gain deeper knowledge and insight into the circuit.

In summary, the study provides evidence that malfunction of neural circuits in the amygdala—the emotion center of the brain—contributes to ABA development, and demonstrates that amygdala circuits is likely a more relevant and robust therapeutic target to treat AN. It also suggests a multi-origin possibility for ABA development, which suggests that future strategy in treating AN requires consideration of combining multiple factors or targeting multiple brain regions.

1. A method of treating or preventing one or more eating disorders, comprising administering to a subject in need thereof an effective amount of electric stimulation (a) to reduce or inhibit the activity of CeA PKC-δ neurons and ovBNST PKC-δ neurons in the brain of the subject or (b) to increase or excite the activity of the brain regions downstream of CeA PKC-δ neurons and ovBNST PKC-δ neurons of the subject.

2. The method of claim 1, wherein an effective amount of electric stimulation is applied to the subject to increase or excite the activity of the brain regions downstream of CeA PKC-δ neurons and ovBNST PKC-δ neurons of the subject.

3. The method of claim 2, wherein the brain regions downstream of CeA PKC-δ neurons and ovBNST PKC-δ neurons are selected from the group consisting of medial part of CeA, ventral lateral part of BNST, and combinations thereof.

4. A method of treating or preventing one or more eating disorders, comprising administering to a subject in need thereof an effective amount of electric stimulation (a) to increase or excite the activity of one or more brain regions upstream of CeA PKC-δ neurons and ovBNST PKC-δ neurons of the subject or (b) to reduce or inhibit the activity of one or more brain regions upstream of CeA PKC-δ neurons and ovBNST PKC-δ neurons of the subject.

5. The method of claim 4, wherein the one or more brain regions upstream of CeA PKC-δ neurons and ovBNST PKC-δ neurons send inhibitory inputs to PKC-δ neurons.

6. The method of claim 5, wherein the one or more brain regions upstream of CeA PKC-δ neurons and ovBNST PKC-δ neurons are arcuate nucleus.

7. (canceled)

8. The method of claim 4, wherein the one or more brain regions upstream of CeA PKC-δ neurons and ovBNST PKC-δ neurons send excitatory inputs to PKC-δ neurons.

9. The method of claim 8, wherein the one or more brain regions upstream of CeA PKC-δ neurons and ovBNST PKC-δ neurons is selected from the group consisting of the insular cortex, piriform cortex, and entorhinal cortex, and combinations thereof.

10. A method of treating or preventing one or more eating disorders, comprising administering to a subject in need thereof an effective amount of a first pharmacological drug (a) to reduce or inhibit the activity of CeA PKC-δ neurons and ovBNST PKC-δ neurons in the brain of the subject, (b) to increase or excite the activity of one or more brain regions downstream of CeA PKC-δ neurons and ovBNST PKC-δ neurons of the subject, (c) to increase or excite the activity of one or more brain regions upstream of CeA PKC-δ neurons and ovBNST PKC-δ neurons of the subject, or (d) to reduce or inhibit the activity of one or more brain regions upstream of CeA PKC-δ neurons and ovBNST PKC-δ neurons of the subject.

11. The method of claim 10, wherein the first pharmacological drug can inhibit the activity of CeA PKC-δ neurons or ovBNST PKC-δ neurons, or both.

12. The method of claim 10, wherein the first pharmacological drug can inhibit the activity of CeA PKC-δ neurons, and wherein the method further comprises administering a second pharmacological drug can inhibit the activity of ovBNST PKC-δ neurons, wherein the first pharmacological drug and the second pharmacological drug are administered simultaneously or sequentially.

13. The method of claim 10, wherein the first pharmacological drug is administered locally to the brain, optionally, wherein the first pharmacological drug is administered locally to the CeA PKC-δ neurons and/or ovBNST PKC-δ neurons in the brain.

14. (canceled)

15. The method of claim 10, wherein the one or more brain regions downstream of CeA PKC-δ neurons and ovBNST PKC-δ neurons are selected from the group consisting of the medial part of CeA, ventral lateral part of BNST, and a combination thereof.

16. (canceled)

17. The method of claim 10, wherein the one or more brain regions upstream of CeA PKC-δ neurons and ovBNST PKC-δ neurons send inhibitory inputs to PKC-δ neurons.

18. The method of claim 17, wherein the one or more brain regions upstream of CeA PKC-δ neurons and ovBNST PKC-δ neurons is in the arcuate nucleus.

19. (canceled)

20. The method of claim 10, wherein the one or more brain regions upstream of CeA PKC-δ neurons and ovBNST PKC-δ neurons send excitatory inputs to PKC-δ neurons.

21. The method of claim 20, wherein the one or more brain regions upstream of CeA PKC-δ neurons and ovBNST PKC-δ neurons is selected from the group consisting of the insular cortex, piriform cortex, and entorhinal cortex, and combinations thereof.

22. The method of claim 1, wherein the one or more eating disorders is selected from the group consisting of anorexia nervosa, bulimia nervosa, and binge-eating disorders.

23. The method of claim 4, wherein the one or more eating disorders is selected from the group consisting of anorexia nervosa, bulimia nervosa, and binge-eating disorders.

24. The method of claim 10, wherein the one or more eating disorders is selected from the group consisting of anorexia nervosa, bulimia nervosa, and binge-eating disorders.
